(19) United States
(12) Patent Application Publication
(10) Pub. No.: US 2025/0122527 A1
(43) Pub. Date: Apr. 17, 2025

(54) COVALENTLY COATED ADENO-ASSOCIATED VIRUS VECTOR FOR ITS USE IN GENE THERAPY

(71) Applicants: INSTITUT QUIMIC DE SARRIA CETS FUNDACIO PRIVADA, BARCELONA (ES); ASOCIACIÓN DUCHENNE PARENT PROJECT ESPAÑA, MADRID (ES)

(72) Inventors: SALVADOR BORRÓS GÓMEZ, BARCELONA (ES); MARTA GUERRA REBOLLO, BARCELONA (ES); MARIA STAMPA LÓPEZ-PINTO, BARCELONA (ES); MARIA SOLEDAD MONTOLIO DEL OLMO, MADRID (ES)

(21) Appl. No.: 18/292,157

(22) PCT Filed: Jul. 25, 2022
(86) PCT No.: PCT/EP2022/070768

§ 371 Date: Jan. 25, 2024

(30) Foreign Application Priority Data
Jul. 26, 2021 (EP) 21382690.2

(51) Int. Cl. C12N 15/86; A61K 47/59; A61K 47/69

(52) U.S. Cl. CPC C12N 15/86; A61K 47/593; A61K 47/6901; C12N 2750/14143

(57) ABSTRACT

An adeno-associated virus (AAV) vector particle, wherein the AAV has at least one capsid protein having one N-terminal covalently bond to a poly(beta aminoester) (PBAE), or a pharmaceutically acceptable salt thereof. A pharmaceutical composition of the AAV vector particle, an acid activated PBAE, and an acid activated oligopeptide modified PBAE (OM-PBAE).

This application claims the benefit of European Patent Application EP21382690.2 filed on Jul. 26, 2021.

### TECHNICAL FIELD

The invention relates to the use of acid activated PBAEs for the preparation of safe and efficient delivery of virus-based therapeutic agents in therapy. More particularly, the invention relates to poly(beta-amino ester)s (PBAE) covalently bonded to an adeno-associated virus vector, and to specific methods of treatment using these PBAE-covalently coated adeno-associated virus vector (AAV vector).

### BACKGROUND ART

For diseases caused by alterations in a single gene, a highly appealing therapy is to replace the altered gene with a normal one using gene therapy. Considering the low stability of gene material in the extracellular environment due to the presence of degradative nucleases, the use of delivery vectors is a must. Among them, viral vectors confer some advantages as compared to non-viral ones. One of the most clinical promising viral vectors for gene therapy are the AAV vectors.

The main disadvantage of the use of AAV vectors for gene therapy is the host immune system. Although AAV are inefficient at transducing antigen-presenting cells (APCs) and have a low immunogenicity profile, exposure to wild-type AAV results in the activation of the immune system against the virus, with development of both humoral and cellular immunity. Furthermore, the majority of the population possesses residual circulating antibodies against AAV due to early exposure in life. As a result, administration of naked AAV vectors elicits a pronounced immune response that gets amplified during the re-administration of the vectors.

To make these vectors evade the host immune response during systemic administration, the coating of these viral particles with poly(beta aminoesters) (PBAEs) by electrostatic interaction of the cationic oligopeptide modified PBAEs (OM-PBAEs) with the viral capsid has been proposed.

EP3406265B1 discloses complexes of end-modified PBAE polymers with virus-based therapeutic agents, wherein the virus-based therapeutic agent, particularly an adenovirus, is non-covalently linked to the PBAE polymer. Similarly, Brugada (cf. Brugada, P., “Boosting intravenous administration of therapeutic viral vectors using an oligopeptide-modified poly(β-aminoester)s-based coating technology”, Doctoral Thesis, Universitat Ramon Llull, Spain, 2018) discloses the use of terminal modified PBAEs with oligopeptides (OM-PBAEs) as coating agents of non-enveloped viral vectors such as adeno-associated viruses (AAV) and adenoviruses (Ad) by electrostatic interaction of the cationic OM-PBAEs with viral capsids. However, some degree of clustering has been observed indicating that further improvements in the technology or coating process are needed in order to produce single coated viral particle suspensions.

Therefore, there is still the need of finding new methods that allow preparing coated viral vectors formulations that are stable while providing the required efficacy and safety.

### SUMMARY OF INVENTION

The inventors have developed a new strategy to coat AAV vectors based on PBAEs that allows obtaining AAV vectors having a higher stability when injected intravenously, thus providing an increased transfection of the target cell.

Unlike in the case of complexes with adenovirus (i.e., adenovirus electrostatically coated with PBAEs), electrostatic coating of AAV vectors with PBAEs failed to increase the stability of the complex or to decrease the presence of neutralizing antibodies against the virus. Without wishing to be bound by theory, the inventors hypothesized that the small size of the AAV (20 nm) made that the charge density of the nanoparticle was not enough to maintain the electrostatic coating, making the covering non-stable once it was injected intravenously.

Therefore, the inventors have synthesized new PBAEs, such as OM-PBAEs, with activated acid residues in the lateral chains capable of covalently bond to the amino groups of the viral capsid proteins through an amidation reaction. Surprisingly, the new particles of polymers covalently bonded to the AAV vectors allowed obtaining stable formulations in the form of dispersed nanoparticles. Additionally, the in vitro and in vivo assays demonstrated the ability of the covalent coating to protect against neutralizing antibodies and that the covalently coated AAV vectors where stable once intravenously injected.

As shown by the results in the examples, the new covalently coated AAV particles were able to transduce different muscular cell lines surprisingly better than the not covalently coated viral particles and the coating efficiently protected from neutralizing antibodies against AAV.

Thus, a first aspect of the present invention relates to an AAV vector particle, wherein the AAV has at least one capsid protein having one N-terminal covalently bond to a poly(beta aminoester) (PBAE), or a pharmaceutically acceptable salt thereof.

In a second aspect, the present invention provides a pharmaceutical composition comprising the AAV vector particle as defined herein above or below, together with a pharmaceutically acceptable vehicle.

In a third aspect, the present invention provides an acid activated PBAE of Formula IIIa as defined herein below.

In a fourth aspect, the present invention provides an acid activated OM-PBAE of formula Ia as defined herein below.

As disclosed herein below, the acid activated PBAE is susceptible of being covalently attached to the AAV vector.

According to a further aspect of the invention, there is provided an AAV vector covalently coated with a PBAE or a composition as defined herein above and below for use in medicine.

According to a further aspect of the invention, there is provided an AAV vector covalently coated with a PBAE or a composition as defined herein for use in systemic viral gene therapy.

According to a further aspect of the invention, there is provided an AAV vector covalently coated with a PBAE or a composition as defined herein for use in the treatment of cancer.

In some embodiments the cancer is liver cancer. In some embodiments the cancer is pancreatic cancer.

According to a further aspect of the invention, there is provided an AAV vector covalently coated with a PBAE or a composition as defined herein for use as a viral vector vaccine in the prophylactic treatment of coronavirus disease 2019 (COVID-19) caused by severe acute respiratory syndrome coronavirus 2 (SARS-CoV-2) or variants thereof.

### BRIEF DESCRIPTION OF DRAWINGS

FIG. 1 illustrates the covalent coupling of a PBAE polymer in the form of an acid activated PBAE (P-L-O-Act) on the capsid of an AAV vector via the N-terminals (primary amino groups) of the capsid proteins.

P is a radical of the PBAE, which is attached to the linker L through an ester or an amide bond; L is a linker which is a biradical having two carbonyl (—CO—) groups, one of them forming the ester o amide bond with the PBAE and the other forming an amide group with N-terminal of the capsid proteins.

FIG. 2 shows the physicochemical characterization of the polymers. A) Changes in the Z potential produced by an increasing ratio μg PBAE/viral particles (vp). The negative surface charge of naked AAVs turns to positive by increasing the amount of coating polymer, the dotted line denotes the change from negative (non-coated) to positive (coated). B) Z potential measurements of increasing concentrations of C6-Act-PEG-CR3 prepared at a 1E-8 ratio μg PBAE/VP. The positive surface charge slightly decreases when increasing concentrations of the PEG polymer were added.

FIG. 3 show the transduction efficiency of AAV particles without coating, with electrostatic or with covalent coating. The studies were done in (A) NSC-34 cell line, (B) C2C12 cell line and (C) MOVA cell line. GFP expression was determined by flow cytometry after 72 h of the viral particle's transduction. Each bar represents the mean+/−SD. P value <0.0001 (***), p value <0.001 (**).

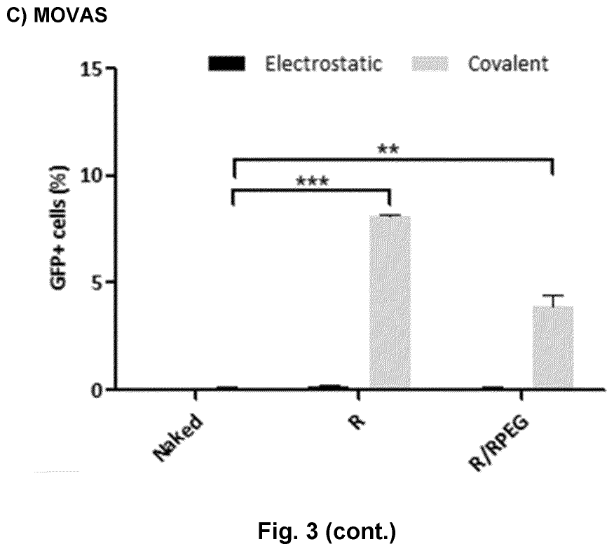

FIG. 4 shows the average transduction efficiency results (% GFP) of the in vitro neutralization assay with sera collected on week 4 (see the scheme above the graph) of the second in vivo study (n=4). Control+(Naked) contains AAV and Hepes, whereas Naked sera (R1-R4), R sera (R5-R8) and R/RPEG sera (R9-R12) contain the serum dilutions (1/1520, 1/2280, 1/3420 and 1/5130) that were incubated with AAV9-GFP and Hepes buffer for 30 minutes at room temperature. Naked, R and R/RPEG groups indicate the average of the 4 mice from each group. Each bar represents the mean+/−SD. P value <0.001 (**).

### DETAILED DESCRIPTION OF THE INVENTION

All terms as used herein in this application, unless otherwise stated, shall be understood in their ordinary meaning as known in the art. Other more specific definitions terms as used in the present application are as set forth below and are intended to apply uniformly throughout the specification and claims unless an otherwise expressly set out definition provides a broader definition.

It is noted that, as used in this specification and the appended claims, the singular forms “a”, “an”, and “the” include plural referents unless the context clearly dictates otherwise.

The term “AAV vector covalently coated with a PBAE”, “PBAE-covalently coated AAV vector”, or simply “covalently coated AAV vector”, as used herein, refers to the AAV vector particle as defined herein above and below, that is to the product obtained by reaction of the amino groups of the viral capsid proteins of an AAV vector with an acid activated PBAE (i.e. a PBAE having an activated carboxyl group), particularly with an acid activated OM-PBAE. The term “covalently coated” is used herein as opposed to the term “complex” in relation to complexes of PBAEs with AAV vectors, i.e., of AAV vectors electrostatically coated of with PBAEs.

As used herein, the term “activated carboxyl group” refers to a carboxyl group in which the hydroxyl group has been replaced by a carboxyl activating group. As used herein, the term “carboxyl activating group” means a group that modifies a carboxyl group to be susceptible to react later on, for instance, to form an amide group with an amino group of a viral capsid proteins. Commonly, a carboxyl activating group is an electron withdrawing moiety that substitutes the hydroxyl moiety of a carboxyl group. Such electron withdrawing moiety enhances polarization and thereby the electrophilicity at the carbonyl carbon.

As mentioned above, a first aspect of the present invention relates to an AAV vector covalently coated with a PBAE, namely to an AAV vector particle, wherein the AAV has at least one capsid protein having one N-terminal covalently bond to a poly(beta aminoester) (PBAE), or a pharmaceutically acceptable salt thereof.

In an embodiment, the PBAE is an oligopeptide modified PBAE (OM-PBAE).

PBAEs can be prepared, for instance, as disclosed in Green J J, Langer R, Anderson D G. “A combinatorial polymer library approach yields insight into nonviral gene delivery”. Acc Chem Res. 2008; 41(6):749-759 and Green J J, Zhou B Y, Mitalipova M M, et al. “Nanoparticles for gene transfer to human embryonic stem cell colonies”. Nano Lett. 2008; 8(10):3126-3130. Particularly, OM-PBAEs can be prepared, for instance, as disclosed in EP3406265.

The PBAE has a reactive hydroxyl or an amine moiety which is linked to the AAV vector though a dicarboxylic acid acting as a linker, wherein one of the carboxylic acid moieties of the dicarboxylic acid is bonded to the hydroxyl or the amine moiety of the PBAE and the other carboxyl group is bonded to the amino groups the viral capsid proteins.

### PBAE Having a Reactive Hydroxyl or Amine Moiety

In an embodiment, the PBAE having a reactive hydroxyl or amine moiety is a polymer of formula III:

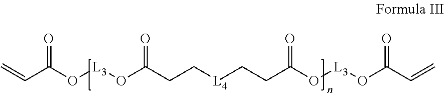

wherein
L₃ is independently selected from the group consisting of alkylene, alkenylene, heteroalkylene, heteroalkenylene, arylene, and heteroarylene; or at least one occurrence of L₃ is

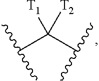

wherein T₁ is

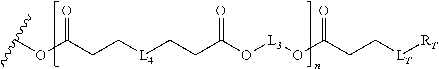

and T₂ is selected from H, alkyl or

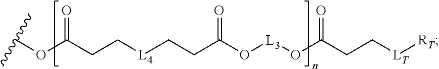

wherein L_{T} is independently selected from the group consisting of:

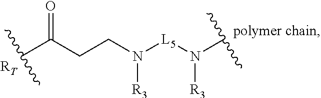

O, S, NRₓ and a bond, wherein Rₓ is independently selected from the group consisting of hydrogen, halogen, alkyl, cycloalkyl, alkenyl, cycloalkenyl, heteroalkyl, heterocycloalkyl, acyl, aryl, and heteroaryl, and the remaining L₃ groups are independently selected at each occurrence from the group consisting of alkylene, alkenylene, heteroalkylene, heteroalkenylene, arylene, and heteroarylene;
L₄ is independently selected from the group consisting of

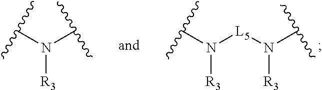

L₅ is independently selected from the group consisting of alkylene, alkenylene, heteroalkylene, heteroalkenylene, arylene, and heteroarylene;
R_{T} is independently selected from an oligopeptide and R_{y};
and wherein R_{y} is selected from the group consisting of hydrogen, halogen, alkyl, cycloalkyl, alkenyl, cycloalkenyl, heteroalkyl, heterocycloalkyl, acyl, aryl, and heteroaryl; each R₃ is independently selected from the group consisting of hydrogen, halogen, alkyl, cycloalkyl, alkenyl, cycloalkenyl, heteroalkyl, heterocycloalkyl, acyl, aryl, heteroaryl, and polyalkylene glycols, wherein said polyalkylene glycol is either bound directly to the nitrogen atom to which R₃ is attached or bound to the nitrogen atom to which R₃ is attached via a linker moiety, wherein said linker moiety is an alkylene, cycloalkylene, alkenylene, cycloalkenylene, heteroalkylene, heterocycloalkylene, arylene or heteroarylene group; provided that at least one R₃ is a moiety having a hydroxyl group (—OH) or an amino group (—NH₂) selected from the group consisting of —(CH₂)ₚNH₂, —(CH₂)ₚOH, —(CH₂)₂(OCH₂CH₂)_{q}NH₂, and —(CH₂)₂(OCH₂CH₂)_{q}OH; and
n is an integer from 5 to 1,000, p is an integer from 1 to 20 (particularly, from 1 to 5), and q is an integer from 1 to 10 (particularly, from 1 to 5);
or a pharmaceutically acceptable salt thereof.

It is understood that L_{T} is a biradical, i.e., an entity with two independent radical centres, and thus, biradical L_{T} connects with R_{T} at one radical centre and with the polymer chain at the other radical centre. Thus, in the structure above defining L_{T}, it is understood that the “R_{T}” and “polymer chain” are depicted to establish the orientation of the biradical L_{T}, i.e. to specify which radical centre connects with R_{T} and which radical centre connect with the polymer chain.

In an embodiment of the PBAE having a reactive hydroxyl or amine moiety, the at least one R₃ is selected from —(CH₂)ₚNH₂ and —(CH₂)ₚOH, wherein p is an integer from 1 to 5, and q is an integer from 1 to 5.

The terms “reactive hydroxyl group” and “reactive amino group” refer to the hydroxyl group or amino group that will form an ester or an amide group with the activated dicarboxylic acid in order to form the acid activated PBAE.

### Preparation of the PBAE Having a Reactive Hydroxyl or Amine Moiety

The polymers of Formula III may be prepared by the reaction of diacrylate monomers of Formula II with substituted amines of formula L₄H₂

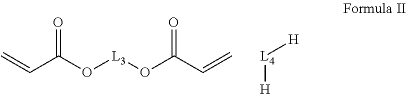

wherein L₃ and L₄ are as defined herein above or below.

The polymers of Formula III wherein at least one R₃ group is a polyalkylene glycol moiety may be prepared in an analogous way by the reaction of diacrylate monomers of Formula II with substituted amines of formula L₄H₂ where the amines are substituted with a polyalkylene glycol moiety optionally bound to the nitrogen of the amine through a linker moiety as defined above.

L_{T} is selected to facilitate coupling of the end-modifying group R_{T} to the PBAE polymer. L_{T} may be a bond (i.e., the carbon atom to which L_{T} is joined in the chemical structures above and below may be directly linked to the R_{T} group), for example where the end-modifying group is an oligopeptide that comprises a terminal cysteine residue.

Rₓ may be independently selected from the group consisting of hydrogen, alkyl, cycloalkyl, alkenyl, cycloalkenyl, heteroalkyl, and heterocycloalkyl, for example, from the group consisting of hydrogen, alkyl, and cycloalkyl.

In compounds disclosed herein above and below where a repeating unit is depicted (by square brackets), each group (e.g., L₃, L₄) within the square brackets is independently selected from the provided definitions for each single repeating unit. In other words, the repeating units within a particular polymer need not be identical.

### Acid Activated PBAE

As mentioned above, in order to be able to covalently bond the PBAE to the AAV vector, an acid activated derivative of the PBAE is previously obtained.

Thus, an aspect of the invention relates to an acid activated PBAE which is a polymer of formula IIIa:

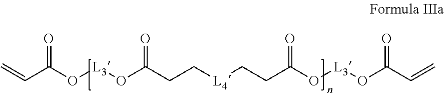

wherein L₃′ is independently selected from the group consisting of alkylene, alkenylene, heteroalkylene, heteroalkenylene, arylene, and heteroarylene; or at least one occurrence of L₃′ is

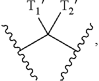

wherein T₁′ is

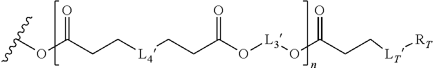

and T₂′ is selected from H, alkyl or

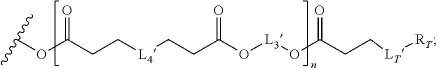

wherein L_{T}′ is independently selected from the group consisting of:

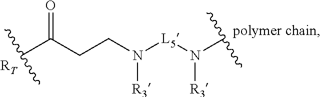

O, S, NRₓ and a bond, wherein Rₓ is independently selected from the group consisting of hydrogen, halogen, alkyl, cycloalkyl, alkenyl, cycloalkenyl, heteroalkyl, heterocycloalkyl, acyl, aryl, and heteroaryl,
and the remaining L₃′ groups are independently selected at each occurrence from the group consisting of alkylene, alkenylene, heteroalkylene, heteroalkenylene, arylene, and heteroarylene;
L₄′ is independently selected from the group consisting of

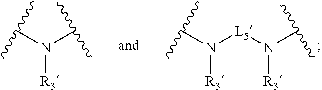

L₅′ is independently selected from the group consisting of alkylene, alkenylene, heteroalkylene, heteroalkenylene, arylene, and heteroarylene;
R_{T} is independently selected from an oligopeptide and R_{y};
and wherein R_{y} is selected from the group consisting of hydrogen, halogen, alkyl, cycloalkyl, alkenyl, cycloalkenyl, heteroalkyl, heterocycloalkyl, acyl, aryl, and heteroaryl;
each R₃′ is independently selected from the group consisting of hydrogen, halogen, alkyl, cycloalkyl, alkenyl, cycloalkenyl, heteroalkyl, heterocycloalkyl, acyl, aryl, heteroaryl, and polyalkylene glycols, wherein said polyalkylene glycol is either bound directly to the nitrogen atom to which R₃′ is attached or bound to the nitrogen atom to which R₃′ is attached via a linker moiety, wherein said linker moiety is an alkylene, cycloalkylene, alkenylene, cycloalkenylene, heteroalkylene, heterocycloalkylene, arylene or heteroarylene group; provided that at least one R₃′ is a moiety selected from the group consisting of —(CH₂)ₚNH—C(═O)-L′-C(═O)—O-Act′, —(CH₂)ₚO—C(═O)-L′-C(═O)—O-Act′, —(CH₂)₂(OCH₂CH₂)_{q}NH—C(═O)-L′-C(═O)—O-Act′, and —(CH₂)₂(OCH₂CH₂)_{q}O—C(═O)-L′-C(═O)—O-Act′, wherein the moiety —C(═O)—O-Act is an activated carboxyl group and Act is an electron withdrawing moiety, or, alternatively, at least one R₃′ is a moiety selected from the group consisting of —(CH₂)ₚNH—C(═O)-L′-C(═O)-Act′, —(CH₂)ₚO—C(═O)-L′-C(═O)-Act′, —(CH₂)₂(OCH₂CH₂)_{q}NH—C(═O)-L′-C(═O)-Act′, and —(CH₂)₂(OCH₂CH₂)_{q}O—C(═O)-L′-C(═O)-Act′, wherein the moiety —C(═O)-Act′ is an activated carboxyl group and Act′ is an electron withdrawing moiety; wherein L′ is a biradical selected from the group consisting of alkylene, cycloalkylene, alkenylene, cycloalkenylene, heteroalkylene, heterocycloalkylene, arylene, and heteroarylene group; and
n is an integer from 5 to 1,000, p is an integer from 1 to 20 (particularly, from 1 to 5), and q is an integer from 1 to 10 (particularly, from 1 to 5);
or a pharmaceutically acceptable salt thereof.

In another embodiment of the compound of formula IIIa, the at least one R₃′ is independently selected from —(CH₂)ₚNH—C(═O)-L′-C(═O)—O-Act and —(CH₂)ₚO—C(═O)-L′-C(═O)—O-Act, wherein p is an integer from 1 to 5, and q is an integer from 1 to 5, and wherein L′ is as defined above.

Particularly, in the embodiments above, L′ is a biradical selected from the group consisting of —(CH₂)ᵣ—, wherein r is an integer independently selected from 1 to 5, particularly r is 2.

Also particularly, in the embodiments above, Act and Act′ are radicals derived from a carboxyl group activating agent and it is formed after the reaction of the carboxyl group activating agent with the carboxyl group to be activated. Suitable carboxyl group activating agents are as defined herein below. Thus, in a particular embodiment, Act is a radical selected from the group consisting of N-succinimidyl, sulfo-N-succinimidyl, N-phthalimidyl, benzotriazolyl, p-chlorotetrafluorophenyl, and pentafluorophenyl. In another embodiment, Act′ is halide (such as chloride, iodide, and fluoride), isothiocyanate, thioester, triazine, pyridinium, and phosphorus derived salts.

### Preparation of the Acid Activated PBAE

The acid activated PBAE molecule can be obtained by coupling an activated acid moiety to the PBAE having a reactive hydroxyl or amine moiety, for instance through an ester or an amide bond with the reactive hydroxyl or amine moiety.

Particularly, the acid activated PBAE can be obtained by a process comprising the following steps:
a) providing a PBAE having a reactive hydroxyl or an amino group;
b) reacting the PBAE with an activated dicarboxylic acid having a first free carboxyl group and a second activated carboxyl group, wherein the first free carboxyl group is bonded to the hydroxyl or the amino group of the PBAE through an esterification or an amidation reaction, respectively, in order to obtain an acid activated PBAE.

In case R_{T} is an oligopeptide, it will be incorporated after activation of the acid, and will be prepared by conventional techniques known to those skilled in the art, wherein the reaction will depend on the amino acid of the oligopeptide involved in the linkage.

The dicarboxylic acid is a compound of formula HO—C(═O)-L′-C(═O)—OH having a first carboxyl group and a second carboxyl group, wherein L′ is a biradical selected from the group consisting of alkylene, cycloalkylene, alkenylene, cycloalkenylene, heteroalkylene, heterocycloalkylene, arylene, and heteroarylene group. Particularly, wherein L′ is a biradical selected from the group consisting of —(CH₂)ᵣ—, wherein r is an integer independently selected from 1 to 5, particularly r=2. Examples of dicarboxylic acids include, without being limited to succinic acid, glutaric acid, adipic acid, and malonic acid.

In an embodiment, the activated dicarboxylic acid can be obtained by reacting a dicarboxylic acid (having a first carboxyl group and a second carboxyl group) with a suitable carboxyl group activating agent as defined above.

Suitable carboxyl group activating agents (also referred to herein as “activators”) include, but are not limited to, N-hydroxysuccinimide (NHS), sulfo-NHS, N-hydroxyphthalimide, hydroxybenzotriazole, pentafluorophenol, di-(p-chlorotetrafluorophenyl)carbonate, di-pentafluorophenyl carbonate, pentafluorophenyl esters, NHS esters, acyl halides (such as acyl chloride, acyl fluoride, acyl azides, anhydrides, carbodiimides, acylimidazoles, acylbenzotriazoles, phosphonium salts, aminium/uranium salts, organophosphorus reagents, organosulfur reagents, triazine coupling reagents, pyridinium coupling reagents, polymer-supported reagents. Particularly, the carboxyl group activating agent is selected from the group consisting of N-hydroxysuccinimide (NHS), sulfo-NHS, N-hydroxyphthalimide, hydroxybenzotriazole, pentafluorophenol, di-(p-chloro tetrafluorophenyl)carbonate, and di-pentafluorophenyl carbonate, pentafluorophenyl esters, NHS esters. As known in the art these activators form active esters. Preferably, the activator is NHS.

As an instance, when the activating agent is NHS a dicarboxylic acid derivative of the NHS is obtained wherein the first of the carboxylic acids is a free carboxyl group and the second carboxyl group is forming an ester with the NHS.

A common way to synthesize an NHS-activated acid is to mix NHS with the desired dicarboxylic acid in the presence of an organic base in an anhydrous solvent. A coupling reagent such as dicyclohexylcarbodiimide (DCC) or ethyl(dimethylaminopropyl) carbodiimide (EDC) is then added to form a highly reactive activated acid intermediate. NHS reacts to form a less labile activated acid. The skilled person will know the reaction conditions required in order to form the ester with NHS of only one of the two carboxylic acids of the dicarboxylic acid. Such esters with an acid and NHS are stable enough to be purified and stored at low temperatures in the absence of water and, as such, are commercially available.

As described above, the first carboxyl group (that is the free carboxyl group, i.e., the one that is not forming an ester with NHS), will be subsequently reacted with the amino group of the PBAE through an amidation reaction, or to the hydroxyl group of the PBAE through an esterification with the use of a coupling reagent in order to obtain an acid activated PBAE.

In order to perform the amidation reaction a wide variety of coupling reagents can be used to convert the free carboxylic acid in a highly reactive activated acid intermediate. Examples of coupling agents are anhydrides, and carbodiimides such as dicyclohexylcarbodiimide (DCC), diisopropylcarbodiimide (DIC), or ethyl(dimethylaminopropyl) carbodiimide (EDC), for instance, in the presence of 4-dimethylaminopyridine (DMAP) or hydroxybenzotriazole (HOBt) as catalyst.

Additionally, additives can be used to improve the efficiency of the reaction. As an instance, in order to avoid the racemization of amino acids by the use of carbodiimides, phosphonium and aminium reagents can be used as coupling reagents.

Examples of aminium reagents include, without being limited to:

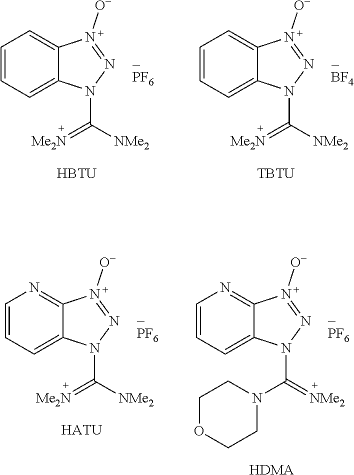

Examples of phosphonium reagents include, without being limited to:

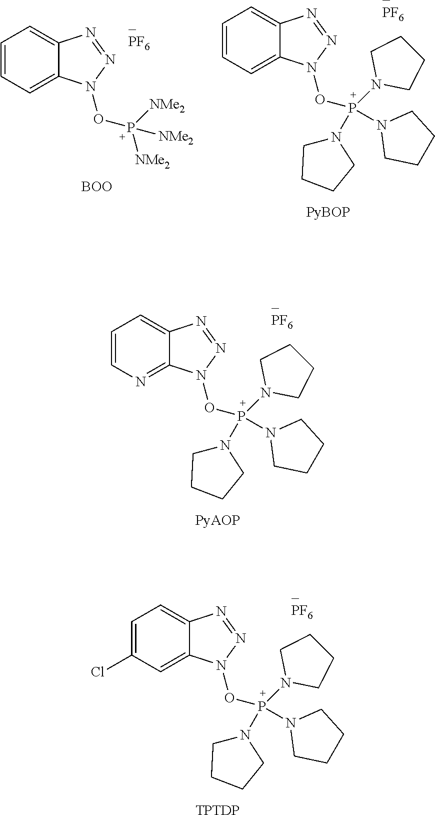

Identically to the amidation reaction, to perform the esterification reaction a wide variety of coupling reagents can be used. Examples of coupling agents are carbodiimides such as dicyclohexylcarbodiimide (DCC), diisopropylcarbodiimide (DIC), or ethyl(dimethylaminopropyl) carbodiimide (EDC), for instance, in the presence of 4-dimethylaminopyridine (DMAP) or hydroxybenzotriazole (HOBt) as catalyst.

### Acid Activated OM-PBAE

Another aspect of the invention relates to acid activated OM-PBAE which is a polymer of Formula Ia:

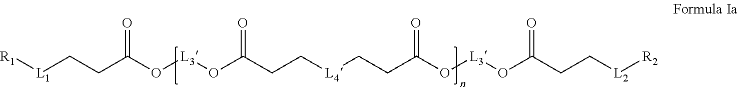

wherein L₃′ is independently selected from the group consisting of alkylene, alkenylene, heteroalkylene, heteroalkenylene, arylene, and heteroarylene; or at least one occurrence of L₃′ is

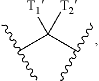

wherein T₁′ is

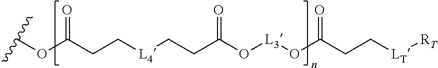

and T₂′ is selected from H, alkyl or

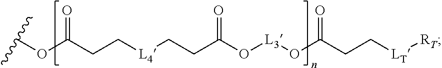

wherein L_{T}′ is independently selected from the group consisting of:

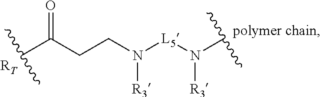

O, S, NRₓ and a bond, wherein Rₓ is independently selected from the group consisting of hydrogen, halogen, alkyl, cycloalkyl, alkenyl, cycloalkenyl, heteroalkyl, heterocycloalkyl, acyl, aryl, and heteroaryl,
and the remaining L₃′ groups are independently selected at each occurrence from the group consisting of alkylene, alkenylene, heteroalkylene, heteroalkenylene, arylene, and heteroarylene; and
L₄′, L₅′, R₃′, and n are as defined above for polymer of Formula IIIa;
each L, and L₂ are independently selected from the group consisting of:

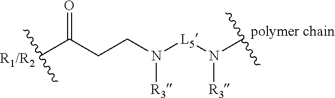

O, S, NRₓ and a bond; wherein Rₓ is independently selected from the group consisting of hydrogen, halogen, alkyl, cycloalkyl, alkenyl, cycloalkenyl, heteroalkyl, heterocycloalkyl, acyl, aryl, and heteroaryl; each R₃″ is independently selected from the group consisting of hydrogen, halogen, alkyl, cycloalkyl, alkenyl, cycloalkenyl, heteroalkyl, heterocycloalkyl, acyl, aryl, heteroaryl, and polyalkylene glycols, wherein said polyalkylene glycol is either bound directly to the nitrogen atom to which R₃″ is attached or bound to the nitrogen atom to which R₃″ is attached via a linker moiety, wherein said linker moiety is an alkylene, cycloalkylene, alkenylene, cycloalkenylene, heteroalkylene, heterocycloalkylene, arylene or heteroarylene group; and
R₁ and R₂ and R_{T} are independently selected from an oligopeptide and R_{y}; wherein at least one of R₁ and R₂ and R_{T} is an oligopeptide; wherein R_{y} is as defined above.

It is understood that L, and L₂ are biradicals, and thus, biradical L, connects with R₁ at one radical centre and with the polymer chain at the other radical centre, and that biradical L₂ connects with R₂ at one radical centre and with the polymer chain at the other radical centre. Thus, it is understood that the “R₁/R₂” and “polymer chain” are depicted in the structure above to establish the orientation of the structure of L₁ and of L₂, i.e., to specify which radical centre connects with R₁ or R₂ and which radical centre connect with the polymer chain.

### Preparation of the Acid Activated OM-PBAE

When the PBAE used to covalently coat the AAV vector is an OM-PBAE, first an acid activated PBAE precursor non-modified with an oligopeptide is obtained as described above, and then it is modified with the oligopeptide in order to obtain the acid activated OM-PBAE, for instance following the process disclosed in EP3406265 for the attachment of an oligopeptide to a PBAE.

The polymer of Formula Ia may be prepared by reaction of the terminal acrylate groups of compound of Formula IIIa with R₁L₁H and R₂L₂H.

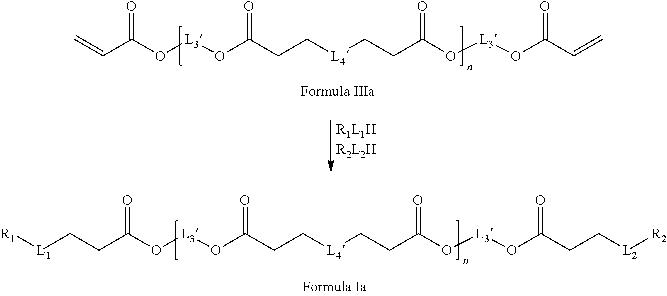

Each L₁ and L₂ is selected to facilitate coupling of the end-modifying groups R₁ and R₂ to the PBAE polymer. Each L, and L₂ may be a bond, for example where the end-modifying group is an oligopeptide that comprises a terminal cysteine residue.

In EP3406265B1 several examples (see for instance Examples 3 to 6) of the reaction of the terminal acrylate groups of compounds similar to the compounds of Formula III with R₁L₁H and R₂L₂H can be found.

In case R_{T} is an oligopeptide, it will be incorporated after activation of the acid, and will be prepared by conventional techniques known to those skilled in the art, wherein the reaction will depend on the amino acid of the oligopeptide involved in the linkage.

### Covalently Coated AAV Vector

As is explained below, the acid activated PBAE (including an OM-PBAE) will be subsequently bonded to an amino group of the viral capsid proteins of the AAV, by reaction with the activated carboxyl group of the PBAE, in order to obtain a covalently coated AAV vector.

Therefore, the hydroxyl reactive group of the at least one R₃ of polymer of formula III as defined above is forming an ester bond with the first carboxyl group of a dicarboxylic acid having a first carboxyl group and a second carboxyl group; or the amine reactive group of the at least one R₃ is forming an amide bond with the first carboxyl group of the dicarboxylic acid; and the second carboxyl group of the dicarboxylic acid is forming an amide group with the amino groups of the viral capsid proteins of the AAV vector has viral capsid proteins having amino groups.

Similarly, the acid activated OM-PBAE will be subsequently bonded to an amino group of the viral capsid proteins of the AAV, by reaction with the activated carboxyl group, in order to obtain a covalently coated AAV vector.

Thus, in another embodiment, the AAV vector particle (i.e. the covalently coated AAV vector), has a structure of Formula (VII)

(A)-(L)-(P) (VII)
wherein:
L is a linker moiety which is a biradical of formula —C(═O)-L′-C(═O)—, wherein L′ is a biradical selected from the group consisting of alkylene, cycloalkylene, alkenylene, cycloalkenylene, heteroalkylene, heterocycloalkylene, arylene, and heteroarylene group.
P is a radical of the PBAE, or of a pharmaceutically acceptable salt thereof, wherein
the PBAE is a polymer of Formula (III),

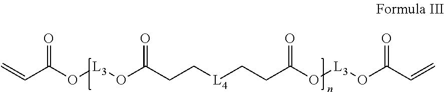

wherein
L₃ is independently selected from the group consisting of alkylene, alkenylene, heteroalkylene, heteroalkenylene, arylene, and heteroarylene; or at least one occurrence of L₃ is

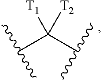

wherein T₁ is

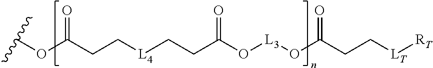

and T₂ is selected from H, alkyl, and

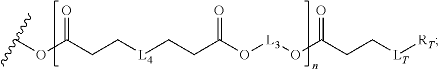

wherein L_{T} is independently selected from the group consisting of:

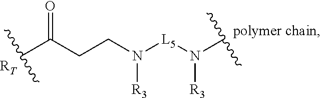

O, S, NRₓ, and a bond, wherein Rₓ is independently selected from the group consisting of hydrogen, halogen, alkyl, cycloalkyl, alkenyl, cycloalkenyl, heteroalkyl, heterocycloalkyl, acyl, aryl, and heteroaryl, and the remaining L₃ groups are independently selected at each occurrence from the group consisting of alkylene, alkenylene, heteroalkylene, heteroalkenylene, arylene, and heteroarylene;
L₄ is independently selected from the group consisting of

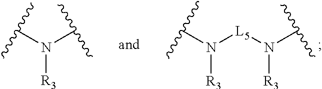

L₅ is independently selected from the group consisting of alkylene, alkenylene, heteroalkylene, heteroalkenylene, arylene, and heteroarylene;
R_{T} is independently selected from an oligopeptide and R_{y};
and wherein R_{y} is selected from the group consisting of hydrogen, halogen, alkyl, cycloalkyl, alkenyl, cycloalkenyl, heteroalkyl, heterocycloalkyl, acyl, aryl, and heteroaryl;
each R₃ is independently selected from the group consisting of hydrogen, halogen, alkyl, cycloalkyl, alkenyl, cycloalkenyl, heteroalkyl, heterocycloalkyl, acyl, aryl, heteroaryl, and polyalkylene glycols, wherein said polyalkylene glycol is either bound directly to the nitrogen atom to which R₃ is attached or bound to the nitrogen atom to which R₃ is attached via a linker moiety, wherein said linker moiety is an alkylene, cycloalkylene, alkenylene, cycloalkenylene, heteroalkylene, heterocycloalkylene, arylene or heteroarylene group;
provided that at least one R₃ is a moiety having a hydroxyl group (—OH) or an amino group (—NH₂) independently selected from the group consisting of —(CH₂)ₚNH₂, —(CH₂)ₚOH, —(CH₂)₂(OCH₂CH₂)_{q}NH₂, and —(CH₂)₂(OCH₂CH₂)_{q}OH;
n is an integer from 5 to 1,000, p is an integer from 1 to 20, and q is an integer from 1 to 10; or alternatively
the PBAE is an OM-PBAE of Formula (I)

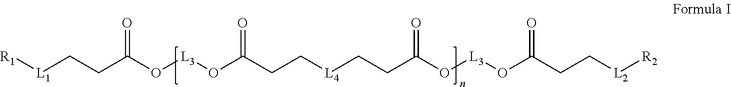

wherein L₃, L₄, and n are as defined above for polymer of Formula III, and
each L₁ and L₂ is independently selected from the group consisting of:

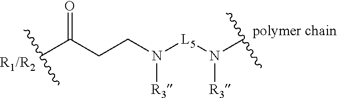

O, S, NRₓ, and a bond; wherein Rₓ is independently selected from the group consisting of hydrogen, halogen, alkyl, cycloalkyl, alkenyl, cycloalkenyl, heteroalkyl, heterocycloalkyl, acyl, aryl, and heteroaryl; each R₃″ is independently selected from the group consisting of hydrogen, halogen, alkyl, cycloalkyl, alkenyl, cycloalkenyl, heteroalkyl, heterocycloalkyl, acyl, aryl, heteroaryl, and polyalkylene glycols, wherein said polyalkylene glycol is either bound directly to the nitrogen atom to which R₃″ is attached or bound to the nitrogen atom to which R₃″ is attached via a linker moiety, wherein said linker moiety is an alkylene, cycloalkylene, alkenylene, cycloalkenylene, heteroalkylene, heterocycloalkylene, arylene or heteroarylene group; and L₅ is as defined above;
R₁ and R₂ and R_{T} are independently selected from an oligopeptide and R_{y}; wherein at least one of R₁ and R₂ and R_{T} is an oligopeptide; wherein R_{y} is as defined above; and
wherein P is attached to the linker L by the hydroxyl or the amino group of the PBAE of Formula (III) or of Formula (I);
A is a radical of an AAV having at least one capsid protein having one N-terminal, wherein A is attached to the linker L by the N-terminal; and wherein
L is attached to the radical P through a bond which is an ester or an amide bond, the ester bond being formed between the —O— of the hydroxyl group of the PBAE of Formula III or of Formula I and one carbonyl group of the linker L; or the amide bond being formed between the —NH— of the amino group of the PBAE of Formula III or of Formula I and one carbonyl group of the linker L; and
L is attached to A through an amide bond formed between the —NH— of the N-terminal of the at least one capsid protein and the other one carbonyl group of the linker L.

In one embodiment of the AAV vector L′ is a biradical selected from the group consisting of —(CH₂)ᵣ—, wherein r is an integer independently selected from 1 to 5, particularly r is 2.

In one embodiment of the AAV vector, the at least one R₃ is independently selected from —(CH₂)ₚNH₂ and —(CH₂)ₚOH, wherein p is an integer from 1 to 5, and q is an integer from 1 to 5.

The present invention further provides a method for the preparation of AAV vectors suitable for use in therapy covalently coated with a PBAE, the method comprising steps of:
providing an AAV vector suitable for use in therapy;
providing an acid activated PBAE as defined herein above; and
mixing the AAV vector and the acid activated PBAE in order to form the AAV vectors covalently coated with a PBAE.

In particular, the AAV vector and the acid activated PBAE as defined herein above may be mixed in solution of Hepes from 10 mM to 40 mM, such as 20 mM, and a pH from 7 to 9 such as of 7.4 at concentrations appropriate to obtain the desired ratio AAV vector/acid activated PBAE and to enable the reaction between the acid activated residues of the PBAEs and the amino groups of the viral capsid proteins and, consequently, to obtain a coating covalently attached to the AAV vector. Thus, to form the AAV vector particles a ratio from 1E-9 to 5E-8 μg pBAE/vp, for example of 1E-8 μg pBAE/vp, is used.

A PBAE-covalently coated AAV vector obtainable by the process as defined above also forms part of the invention. All particular embodiments of the PBAEs or the activated PBAEs (including OM-PBAEs and OM-PBAEs activated PBAEs) are also particular embodiments of the PBAE-covalently coated AAV vector.

As described above, in an embodiment, the AAV vector is covalently coated with an acid activated PBAE comprising or consisting of a polymer of Formula IIIa or of Formula Ia as defined herein above or below, wherein the acid activated group of the PBAE is forming an amide group with the amino groups of the viral capsid proteins.

In another embodiment, the acid activated PBAE is a combination of two or more different polymers of Formula Ia or of Formula IIIa, wherein at least one of the polymers is a PAG-ylated or PEG-ylated polymer of Formula Ia or of Formula IIIa and at least one of the polymers does not contain a PAG or PEG moiety.

In another embodiment, the polymeric material is a combination of two different polymers of Formula Ia or of Formula IIIa as defined herein, wherein one of the polymers is a PEG-ylated polymer and the other one does not contain a PAG or PEG moiety. In another embodiment, the polymeric material comprises or consist of (i) a first polymer of Formula Ia or of Formula IIIa as defined above wherein R₃′ and R₃″ are not a polyalkylene glycol, in combination with (ii) a second polymer for Formula Ia or of Formula IIIa as defined above wherein at least one R₃′ or at least one R₃″ group is a polyalkylene glycol. The ratio of the two different polymers (i) and (ii) may be 99:1, 95:5, 90:10, 75:25, 65:35, 50:50, 35:65, 25:75, 10:90, 5:95, or 1:99 by weight or by volume. In one embodiment the ratio of polymers (i) and (ii) is from 75:25 to 55:45, preferably 65:35 (v/v).

In an embodiment, the AAV vector is covalently coated with a combination of (i) R3C—C6-Act-CR3 and (ii) R3C—C6-Act-CR3-PEG, as disclosed in the Examples below, preferably where polymers (i) and (ii) are in a ratio from 90/10 to 60/40, such as of 65:35.

Examples of the AAV vector include, without being limited to, rAAV9-GPF, AAV1 to AAV12, AAVrh10, AAV LP2-20, and AAV LP2-10.

In an embodiment, the composition comprises the covalently coated AAV vector as defined herein above in an amount from 1% to 90% by weight, or from 5% to 50% by weight, or from 10% to 30% by weight, of the composition. The composition may further comprise a pharmaceutically acceptable vehicle. The pharmaceutically acceptable vehicle may be any pharmaceutically acceptable diluent or excipient, as known in the art. The pharmaceutically acceptable vehicle is typically pharmacologically inactive. Preferably, the pharmaceutically acceptable vehicle is a polar liquid. Particularly preferred pharmaceutically acceptable vehicles include water and physiologically acceptable aqueous solutions containing salts and/or buffers, for example, saline or phosphate-buffered saline. Optionally, the pharmaceutically acceptable vehicle is a biological fluid. A liquid vehicle may be removed by, for example, lyophilization, evaporation or centrifugation for storage or to provide a powder for pulmonary or nasal administration, a powder for suspension for infusion, or tablets or capsules for oral administration.

Administration of the compositions described herein can be via any of the accepted modes of administration for such compositions including, but not limited to, orally, sublingually, subcutaneously, intravenously, intratumorally, intranasally, topically, transdermally, intraperitoneally, intramuscularly, intrapulmonarilly, vaginally, rectally, or intraocularly. In an embodiment, the composition is for oral or parenteral. In another embodiment, the composition is administered intravenously or intratumorally.

The covalently coated AAV vector of the present disclosure is biocompatible and sufficiently resistant to the environment of use that a sufficient amount of the covalently coated AAV vector remains substantially intact after entry into the mammalian body so as to be able to reach the desired target and achieve the desired physiological effect. The polymers described herein are biocompatible and preferably biodegradable.

Herein, the term ‘biocompatible’ describes as substance which may be inserted or injected into a living subject without causing an adverse response. For example, it does not cause inflammation or acute rejection by the immune system that cannot be adequately controlled. It will be recognized that “biocompatible” is a relative term, and some degree of immune response is to be expected even for substances that are highly compatible with living tissue. An in vitro test to assess the biocompatibility of a substance is to expose it to cells; biocompatible substances will typically not result in significant cell death (for example, >20%) at moderate concentrations (for example, 29 μg/10⁴ cells).

Herein, the term ‘biodegradable’ describes a polymer which degrades in a physiological environment to form monomers and/or other non-polymeric moieties that can be reused by cells or disposed of without significant toxic effect. Degradation may be biological, for example, by enzymatic activity or cellular machinery, or may be chemical, typically a chemical process that takes place under physiological conditions. Degradation of a polymer may occur at varying rates, with a half-life in the order of days, weeks, months, or years, depending on the polymer or copolymer used. The components preferably do not induce inflammation or other adverse effects in vivo. In certain preferred embodiments, the chemical reactions relied upon to break down the biodegradable compounds are uncatalysed.

### Oligopeptides

According to the present invention, an “oligopeptide” comprises a string of at least three amino acids linked together by peptide bonds. Such peptides preferably contain only natural amino acids, although non-natural amino acids (i.e., compounds that do not occur in nature but that can be incorporated into a polypeptide chain) and/or amino acid analogues as are known in the art may alternatively be employed. Also, one or more of the amino acids in such peptides may be modified, for example, by the addition of a chemical entity such as a carbohydrate group, a phosphate group, a farnesyl group, an isofarnesyl group, a fatty acid group, or a linker for conjugation, functionalization, or other modification, etc. The oligopeptides in the polymers defined herein typically comprise from 3 to 20 amino acid residues, more preferably from 3 to 10 amino acid residues, more preferably from 3 to 6 amino acid residues. Alternatively, the oligopeptides in the polymers defined herein may comprise from 4 to 20 amino acid residues, more preferably from 4 to 10 amino acid residues, more preferably from 4 to 6 amino acid residues.

In the polymers Formula Ia, of Formula III, of Formula IIIa the or each oligopeptide preferably has a net positive charge at pH 7. The or each oligopeptide may comprise naturally occurring amino acids that are positively charged at pH 7, that is, lysine, arginine and histidine. For example, the or each oligopeptide may be selected from the group consisting of polylysine, polyarginine, and polyhistidine, each of which may be terminated with cysteine.

In a preferred embodiment, optionally in combination with one or more features of the particular embodiments defined above, the or each oligopeptide is a compound of Formula IV:

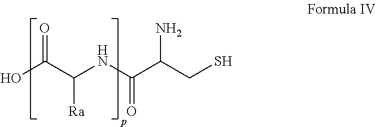

wherein p is an integer from 2 to 19, typically from 3 to 9 or from 3 to 5, and wherein Rₐ is selected at each occurrence from the group consisting of H₂NC(═NH)—NH(CH₂)₃—, H₂N(CH₂)₄—, and (1H-imidazol-4-yl)-CH₂—.

Where the or each oligopeptide is a compound of Formula IV, the L₁ and/or L₂ (and/or L_{T}, when present) linking the or each oligopeptide to the polymer is a bond and the terminal cysteine residue provides a means of coupling the or each oligopeptide to the acrylate terminated compound of Formula III. The thiol functionality provides faster, more efficient and more easily controlled addition to the double bond. By contrast, where the or each oligopeptide is terminated in an amine functionality for coupling, an excess of this compound is required in the coupling step.

In the polymers of Formula Ia, of Formula III, of Formula IIIa the or each oligopeptide may have a net negative charge at pH 7. The or each oligopeptide may comprise naturally occurring amino acids that are negatively charged at pH 7, that is, aspartic acid and glutamic acid. For example, the or each oligopeptide may be selected from polyaspartic acid and polyglutamic acid, each of which may be terminated with cysteine. In this embodiment, the or each oligopeptide may be a compound of Formula IV wherein p is an integer from 2 to 19, typically from 3 to 9 or from 3 to 5, and wherein Rₐ is HO₂C(CH₂)₂— or HO₂C—CH₂—. In this case, the L₁ and/or L₂ linking the or each oligopeptide to the polymer is a bond as the terminal cysteine residue provides a means of coupling the or each oligopeptide to the acrylate terminated intermediate, Formula IV.

Alternatively, the or each oligopeptide may comprise a mixture of naturally occurring amino acids that are negatively charged at pH 7 and naturally occurring amino acids that are positively charged at pH 7.

In the polymers of Formula Ia, of Formula III, of Formula IIIa the or each oligopeptide may be hydrophobic. The or each oligopeptide may comprise naturally occurring amino acids that are hydrophobic such as valine, leucine, isoleucine, methionine, tryptophan, phenylalanine, cysteine, tyrosine and alanine; in particular, the or each oligopeptide may comprise valine, leucine, isoleucine, methionine, tryptophan and phenylalanine.

In the polymers of Formula Ia, of Formula III, of Formula IIIa the or each oligopeptide may be hydrophilic. The or each oligopeptide may comprise naturally occurring amino acids that are hydrophilic such as serine, threonine, cysteine, asparagine and glutamine, and may further comprise naturally occurring amino acids that are charged at pH7.

Thus, an example, the compound of Formula IIIa can be reacted with compounds of Formula IV to form a compound of Formula V:

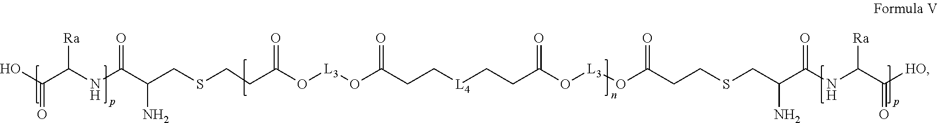

wherein p and Rₐ independently at each occurrence are selected from the lists defined above. In some cases, each occurrence of p is the same and the Rₐ groups are selected such that the sequence of Rₐ groups starting from the sulfur linkage is the same at each end of the compound, that is, p and Rₐ are selected such that the polymer has two-fold symmetry about L₄.

In an alternative to the above step, the compound of Formula IIIa can be reacted with compounds of formula H₂NR_{y}, wherein R_{y} is as defined above, and compounds of Formula IV and the resulting mixture is separated to obtain a compound of Formula VI:

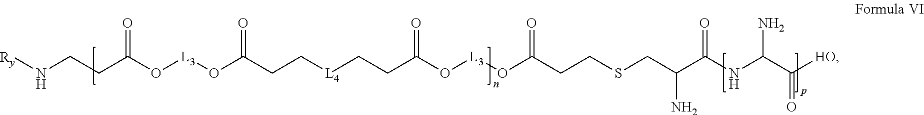

wherein Rₐ is independently selected at each occurrence from the lists defined above and p is as defined above.

It will be recognized that further methods of attaching an oligopeptide to the compound of Formula IIIa would be available to the skilled person, who would be aware of appropriate nucleophiles for reaction at the terminal acrylate groups of Formula IIIa.

### Substituents

In an embodiment, optionally in combination with one or more features of the particular embodiments defined above or below, in the polymers of Formula Ia either both R₁ and R₂ are oligopeptides, for example, wherein R₁ and R₂ are different oligopeptides, or one of R₁ and R₂ is an oligopeptide and one of R₁ and R₂ is R_{y}.

Where one of R₁ and R₂ is R_{y}, then R_{y} is preferably selected from the group consisting of hydrogen, —(CH₂)ₘNH₂, —(CH₂)ₘNHMe, —(CH₂)ₘOH, —(CH₂)ₘCH₃, —(CH₂)₂(OCH₂CH₂)ₘNH₂, —(CH₂)₂(OCH₂CH₂)ₘOH, and —(CH₂)₂(OCH₂CH₂)ₘCH₃ wherein m is an integer from 1 to 20, for example from 1 to 5. Preferably, R_{y} is selected from the group consisting of —(CH₂)ₘNH₂, —(CH₂)ₘNHMe, and —(CH₂)₂(OCH₂CH₂)ₘNH₂. Preferably, when L₁ is NH or NRₓ, and one of R₁ and R₂ is R_{y}, then R_{y} is different to R₃.

The polymers may be asymmetric. For example, in the polymers of Formula Ia, one of R₁ and R₂ may be an oligopeptide and the other may be R_{y}. Alternatively, R₁ and R₂ may each be a different oligopeptide. In polymers where R_{T} is present, at least one selected from R₁, R₂ and the one or two occurrences of R_{T} may be an oligopeptide and the remaining groups selected from R₁, R₂ and the one or two occurrences of R_{T} may be R_{y}. Alternatively, R₁, R₂ and the one or two occurrences of R_{T} may each be a different oligopeptide.

For example, in the polymers of Formula Ia one of R₁ and R₂ may be CysArgArgArg and the other may be derived from H₂N(CH₂)₃CH(CH₃)CH₂NH₂.

In another embodiment, optionally in combination with one or more features of the particular embodiments defined above or below, in the polymers of Formula Ia, of Formula III, or of Formula IIIa, L₃, L₃′, L₅, and L₅′ may be independently selected from alkylene, alkenylene, heteroalkylene or heteroalkenylene and including polyethylene glycol linkers. Said alkylene, alkenylene, heteroalkylene or heteroalkenylene moieties may be of 1-20 carbon atoms, preferably of 1-12 carbon atoms, more preferably of 1-6 carbon atoms. Said polyethylene glycol linkers may be of 3 to 25 atoms in length, preferably of 3 to 18 atoms in length.

In a preferred embodiment, L₃, L₃′, L₅, and L₅′ are independently selected from alkylene moieties, preferably of 1-12 carbon atoms, more preferably of 1-6 carbon atoms, more preferably of 3-5 carbon atoms, and in a preferred embodiment of 4 carbon atoms. In a particularly preferred embodiment, L₃ and L₃′ are selected from —CH₂—, —(CH₂)₂—, —(CH₂)₃—, —(CH₂)₄—, —(CH₂)₅—, and —(CH₂)₆—.

In a further embodiment, optionally in combination with one or more features of the particular embodiments defined above or below, one or more carbon atoms in L₃ and L₃′ and/or L₅ and L₅′ (particularly as defined in the aforementioned preferred embodiments) may be replaced with —S—S—. In this embodiment, L₃ and L₃′ are preferably selected from —(CH₂)_{z}—S—S—(CH₂)_{z}— wherein the value of each z is independently selected from 1 to 4 and preferably from 2 to 3 and preferably 2, preferably wherein the value of each z is the same. The inclusion of at least one disulfide bond in the main polymer chain can facilitate unpacking of the virus-based therapeutic agents inside the target cells. Preferably, L₄ and L₄′ are —N(R₃)—.

Preferably, each R₃, R₃′, and R₃″ are independently selected from the group consisting of hydrogen, —(CH₂)ₚNH₂, —(CH₂)ₚNHMe, —(CH₂)ₚOH, —(CH₂)ₚCH₃, —(CH₂)₂(OCH₂CH₂)_{q}NH₂, —(CH₂)₂(OCH₂CH₂)_{q}OH, —(CH₂)₂(OCH₂CH₂)_{q}CH₃, and polyalkylene glycols, wherein p is an integer from 1 to 20 (preferably 1 to 5), and q is an integer from 1 to 10, for example from 1 to 5, and wherein said polyalkylene glycol is either bound directly to the nitrogen atom to which R₃, R₃′, or R₃″ is attached or bound to the nitrogen atom to which R₃, R₃′, or R₃″ is attached via a linker moiety, wherein said linker moiety is an alkylene, cycloalkylene, alkenylene, cycloalkenylene, heteroalkylene, heterocycloalkylene, arylene, or heteroarylene group; provided that at least one R₃ is selected from the group consisting of —(CH₂)ₚNH₂, —(CH₂)ₚOH, —(CH₂)₂(OCH₂CH₂)_{q}NH₂, and —(CH₂)₂(OCH₂CH₂)_{q}OH; and provided that at least one R₃′ is a moiety selected from the group consisting of —(CH₂)ₚNH—C(═O)-L′-C(═O)—O-Act, —(CH₂)ₚO—C(═O)-L′-C(═O)—O-Act, —(CH₂)₂(OCH₂CH₂)_{q}NH—C(═O)-L′-C(═O)—O-Act, and —(CH₂)₂(OCH₂CH₂)_{q}O—C(═O)-L′-C(═O)—O-Act; wherein the moiety —C(═O)—O-Act and L′ are as defined above.

In another embodiment, optionally in combination with one or more features of the particular embodiments defined above, at least one R₃, R₃′, or R₃″ group is a polyalkylene glycol, preferably a polyethylene glycol. In another embodiment the polyalkylene glycol (for example, polyethylene glycol) is bound directly to the nitrogen atom to which R₃, R₃′, or R₃″ is attached. In another embodiments the polyalkylene glycol (for example, polyethylene glycol) is bound to the nitrogen atom to which R₃, R₃′, or R₃″ is attached via a linker moiety. In another embodiment the linker moiety is an alkylene, alkenylene or heteroalkylene group, preferably an alkylene group. In another embodiment the linker moiety is from 3 to 20 carbon and/or heteroatoms in length, preferably from 4 to 15 carbon and/or heteroatoms in length, more preferably from 5 to 10 carbon and/or heteroatoms in length.

In another embodiment, optionally in combination with one or more features of the particular embodiments defined above, at least one R₃ or R₃′ group is a polyalkylene glycol and the polyalkylene glycol (for example, polyethylene glycol) bound directly to the nitrogen atom of an L₄ or L₄′ group. In some embodiments at least one R₃ or R₃′ group is a polyalkylene glycol and the polyalkylene glycol (for example, polyethylene glycol) bound to the nitrogen atom of an L₄ or L₄′ group via a linker moiety. In preferred embodiments the linker moiety is an alkylene, alkenylene or heteroalkylene group, more preferably the linker moiety is an alkylene group. In some embodiments the linker moiety is from 3 to 20 carbon and/or heteroatoms in length, preferably from 4 to 15 carbon and/or heteroatoms in length, more preferably from 5 to 10 carbon and/or heteroatoms in length.

In another embodiment, optionally in combination with one or more features of the particular embodiments defined above or below, L₃ and L₃′ are independently selected from the group consisting of alkylene, alkenylene, heteroalkylene, heteroalkenylene, arylene, and heteroarylene.

In another embodiment, optionally in combination with one or more features of the particular embodiments defined above or below, at least one occurrence of L₃ is

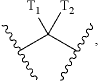

wherein T₁ is

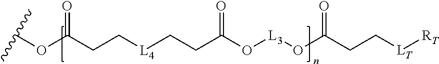

and T₂ is selected from H, alkyl or

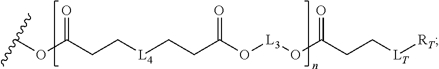

wherein L_{T} is independently selected from the group consisting of:

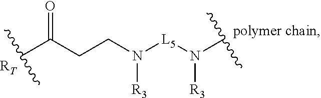

O, S, NRₓ and a bond, wherein Rₓ is independently selected from the group consisting of hydrogen, halogen, alkyl, cycloalkyl, alkenyl, cycloalkenyl, heteroalkyl, heterocycloalkyl, acyl, aryl, and heteroaryl, and the remaining L₃ groups are independently selected at each occurrence from the group consisting of alkylene, alkenylene, heteroalkylene, heteroalkenylene, arylene, and heteroarylene.

In another embodiment, optionally in combination with one or more features of the particular embodiments defined above or below, at least one occurrence of L₃′ is

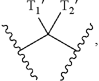

wherein T₁′ is

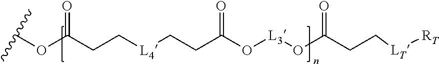

and T₂′ is selected from H, alkyl or

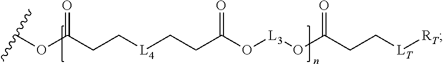

wherein L_{T}′ is independently selected from the group consisting of:

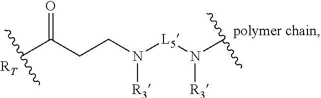

O, S, NRₓ and a bond, wherein Rₓ is independently selected from the group consisting of hydrogen, halogen, alkyl, cycloalkyl, alkenyl, cycloalkenyl, heteroalkyl, heterocycloalkyl, acyl, aryl, and heteroaryl, and the remaining L₃′ groups are independently selected at each occurrence from the group consisting of alkylene, alkenylene, heteroalkylene, heteroalkenylene, arylene, and heteroarylene.

In another embodiment, optionally in combination with one or more features of the particular embodiments defined above or below, the PBAE is a PBAE end-modified with at least one oligopeptide. In another embodiment, optionally in combination with one or more features of the particular embodiments defined above or below, the PBAE is a PBAE substituted with at least one polyalkylene glycol group (preferably a polyethylene glycol group) either directly or through a linker, and end-modified with at least one oligopeptide.

In another embodiment, optionally in combination with one or more features of the particular embodiments defined above or below, in Formula Ia, of Formula III, or of Formula IIIa above, n is preferably from 10 to 700, more preferably from 20 to 500. The molecular weight of the polymer of Formula Ia, of Formula III, or of Formula IIIa is preferably from 500 to 150,000 g/mol, more preferably from 700 to 100,000 g/mol, more preferably from 2,000 to 50,000 g/mol, more preferably from 5,000 to 40,000 g/mol. In embodiments where at least one R₃ group is a polyalkylene glycol (e.g. polyethylene glycol) the molecular weight of the polymer of Formula I or Formula III is preferably from 2,500 to 150,000 g/mol, more preferably from 2,700 to 100,000 g/mol, more preferably from 4,000 to 50,000 g/mol, more preferably from 7,000 to 40,000 g/mol.

Certain polymers of Formula Ia, of Formula III, or of Formula IIIa of the present invention, may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

### Chemical Groups

The term “halogen” (or “halo”) includes fluorine, chlorine, bromine and iodine.

The term “alkyl” includes monovalent, straight or branched, saturated, acyclic hydrocarbyl groups. Alkyl is suitably C₁₋₁₀alkyl, or C₁₋₆alkyl, or C₁₋₄alkyl, such as methyl, ethyl, n-propyl, i-propyl or t-butyl groups. Alkyl may be substituted.

The term “cycloalkyl” includes monovalent, saturated, cyclic hydrocarbyl groups. Cycloalkyl is suitably C₃₋₁₀cycloalkyl, or C₃₋₆cycloalkyl such as cyclopentyl and cyclohexyl. Cycloalkyl may be substituted.

The term “alkoxy” means alkyl-O—.

The term “alkylamino” means alkyl-NH—.

The term “alkylthio” means alkyl-S(O)ₜ—, wherein t is defined below.

The term “alkenyl” includes monovalent, straight or branched, unsaturated, acyclic hydrocarbyl groups having at least one carbon-carbon double bond and, suitably, no carbon-carbon triple bonds. Alkenyl is suitably C₂₋₁₀alkenyl, or C₂₋₆alkenyl, or C₂₋₄alkenyl. Alkenyl may be substituted.

The term “cycloalkenyl” includes monovalent, partially unsaturated, cyclic hydrocarbyl groups having at least one carbon-carbon double bond and, suitably, no carbon-carbon triple bonds. Cycloalkenyl is suitably C₃₋₁₀cycloalkenyl, or C₅₋₁₀cycloalkenyl, e.g. cyclohexenyl or benzocyclohexyl. Cycloalkenyl may be substituted.

The term “alkynyl” includes monovalent, straight or branched, unsaturated, acyclic hydrocarbyl groups having at least one carbon-carbon triple bond and, suitably, no carbon-carbon double bonds. Alkynyl is suitably C₂₋₁₀alkynyl, or C₂₋₆alkynyl, or C₂₋₄alkynyl. Alkynyl may be substituted.

The term “alkylene” includes divalent, straight or branched, saturated, acyclic hydrocarbyl groups. Alkylene is suitably C₁₋₁₀alkylene, or C₁₋₆alkylene, or C₁₋₄alkylene, such as methylene, ethylene, n-propylene, i-propylene or t-butylene groups. Alkylene may be substituted.

The term “alkenylene” includes divalent, straight or branched, unsaturated, acyclic hydrocarbyl groups having at least one carbon-carbon double bond and, suitably, no carbon-carbon triple bonds. Alkenylene is suitably C₂₋₁₀alkenylene, or C₂₋₆alkenylene, or C₂₋₄alkenylene. Alkenylene may be substituted.

The term “heteroalkyl” includes alkyl groups, for example, C₁₋₆₅alkyl groups, C₁₋₁₇alkyl groups or C₁₋₁₀alkyl groups, in which up to twenty carbon atoms, or up to ten carbon atoms, or up to two carbon atoms, or one carbon atom, are each replaced independently by O, S(O)ₜ or N, provided at least one of the alkyl carbon atoms remains. The heteroalkyl group may be C-linked or hetero-linked, i.e. it may be linked to the remainder of the molecule through a carbon atom or through O, S(O)ₜ or N, wherein t is defined below. Heteroalkyl may be substituted.

The term “heterocycloalkyl” includes cycloalkyl groups in which up to ten carbon atoms, or up to two carbon atoms, or one carbon atom, are each replaced independently by O, S(O)ₜ or N, provided at least one of the cycloalkyl carbon atoms remains. Examples of heterocycloalkyl groups include oxiranyl, thiaranyl, aziridinyl, oxetanyl, thiatanyl, azetidinyl, tetrahydrofuranyl, tetrahydrothiophenyl, pyrrolidinyl, tetrahydropyranyl, tetrahydrothiopyranyl, piperidinyl, 1,4-dioxanyl, 1,4-oxathianyl, morpholinyl, 1,4-dithianyl, piperazinyl, 1,4-azathianyl, oxepanyl, thiepanyl, azepanyl, 1,4-dioxepanyl, 1,4-oxathiepanyl, 1,4-oxaazepanyl, 1,4-dithiepanyl, 1,4-thieazepanyl and 1,4-diazepanyl. The heterocycloalkyl group may be C-linked or N-linked, i.e. it may be linked to the remainder of the molecule through a carbon atom or through a nitrogen atom. Heterocycloalkyl may be substituted.

The term “heteroalkenyl” includes alkenyl groups, for example, C₁₋₆₅alkenyl groups, C₁₋₁₇alkenyl groups or C₁₋₁₀alkenyl groups, in which up to twenty carbon atoms, or up to ten carbon atoms, or up to two carbon atoms, or one carbon atom, are each replaced independently by O, S(O)ₜ or N, provided at least one of the alkenyl carbon atoms remains. The heteroalkenyl group may be C-linked or hetero-linked, i.e. it may be linked to the remainder of the molecule through a carbon atom or through O, S(O)ₜ or N. Heteralkenyl may be substituted.

The term “heterocycloalkenyl” includes cycloalkenyl groups in which up to three carbon atoms, or up to two carbon atoms, or one carbon atom, are each replaced independently by O, S(O)ₜ or N, provided at least one of the cycloalkenyl carbon atoms remains. Examples of heterocycloalkenyl groups include 3,4-dihydro-2H-pyranyl, 5-6-dihydro-2H-pyranyl, 2H-pyranyl, 1,2,3,4-tetrahydropyridinyl and 1,2,5,6-tetrahydropyridinyl. The heterocycloalkenyl group may be C-linked or N-linked, i.e. it may be linked to the remainder of the molecule through a carbon atom or through a nitrogen atom. Heterocycloalkenyl may be substituted.

The term “heteroalkynyl” includes alkynyl groups, for example, C₁₋₆₅alkynyl groups, C₁₋₁₇alkynyl groups or C₁₋₁₀alkynyl groups, in which up to twenty carbon atoms, or in which up to ten carbon atoms, or up to two carbon atoms, or one carbon atom, are each replaced independently by O, S(O)ₜ or N, provided at least one of the alkynyl carbon atoms remains. The heteroalkynyl group may be C-linked or hetero-linked, i.e. it may be linked to the remainder of the molecule through a carbon atom or through O, S(O)ₜ or N. Heteroalkynyl may be substituted.

The term “heteroalkylene” includes alkylene groups, for example, C₁₋₆₅alkylene groups, C₁₋₁₇alkylene groups or C₁₋₁₀alkylene groups, in which up to twenty carbon atoms, or in which up to ten carbon atoms, or up to two carbon atoms, or one carbon atom, are each replaced independently by O, S(O)ₜ or N, provided at least one of the alkylene carbon atoms remains. Heteroalkynylene may be substituted.

The term “heteroalkenylene” includes alkenylene groups, for example, C₁₋₆₅alkenylene groups, C₁₋₁₇alkenylene groups or C₁₋₁₀alkenylene groups, in which up to twenty carbon atoms, or in which up to ten carbon atoms, or up to two carbon atoms, or one carbon atom, are each replaced independently by O, S(O)ₜ or N, provided at least one of the alkenylene carbon atoms remains. Heteroalkenylene may be substituted.

The term “aryl” includes monovalent, aromatic, cyclic hydrocarbyl groups, such as phenyl or naphthyl (e.g. 1-naphthyl or 2-naphthyl). In general, the aryl groups may be monocyclic or polycyclic fused ring aromatic groups. Preferred aryl are C₆-C₁₄aryl. Aryl may be substituted.

Other examples of aryl groups are monovalent derivatives of aceanthrylene, acenaphthylene, acephenanthrylene, anthracene, azulene, chrysene, coronene, fluoranthene, fluorene, as-indacene, s-indacene, indene, naphthalene, ovalene, perylene, phenalene, phenanthrene, picene, pleiadene, pyrene, pyranthrene and rubicene.

The term “arylalkyl” means alkyl substituted with an aryl group, e.g. benzyl.

The term “heteroaryl” includes aryl groups in which one or more carbon atoms are each replaced by heteroatoms independently selected from O, S, N, and NR^{N}, where R^{N} is defined below (and in one embodiment is H or alkyl (e.g. C₁₋₆alkyl)). Heteroaryl may be substituted.

In general, the heteroaryl groups may be monocyclic or polycyclic (e.g. bicyclic) fused ring heteroaromatic groups. Typically, heteroaryl groups contain 5-14 ring members (preferably 5-10 members) wherein 1, 2, 3 or 4 ring members are independently selected from O, S, N, and NR^{N}. A heteroaryl group is suitably a 5, 6, 9 or 10 membered, e.g. 5-membered monocyclic, 6-membered monocyclic, 9-membered fused-ring bicyclic or 10-membered fused-ring bicyclic.

Monocyclic heteroaromatic groups include heteroaromatic groups containing 5-6 ring members wherein 1, 2, 3 or 4 ring members are independently selected from O, S, N, and NR^{N}.

5-Membered monocyclic heteroaryl groups may contain 1 ring member which is an —NR^{N}— group, an —O— atom or an —S— atom and, optionally, 1-3 ring members (e.g. 1 or 2 ring members) which are ═N— atoms (where the remainder of the 5 ring members are carbon atoms).

Examples of 5-membered monocyclic heteroaryl groups are pyrrolyl, furanyl, thiophenyl, pyrazolyl, imidazolyl, isoxazolyl, oxazolyl, isothiazolyl, thiazolyl, 1,2,3 triazolyl, 1,2,4 triazolyl, 1,2,3 oxadiazolyl, 1,2,4 oxadiazolyl, 1,2,5 oxadiazolyl, 1,3,4 oxadiazolyl, 1,3,4 thiadiazolyl, pyridyl, pyrimidinyl, pyridazinyl, pyrazinyl, 1,3,5 triazinyl, 1,2,4 triazinyl, 1,2,3 triazinyl and tetrazolyl.

Examples of 6-membered monocyclic heteroaryl groups are pyridinyl, pyridazinyl, pyrimidinyl and pyrazinyl.

6-Membered monocyclic heteroaryl groups may contain 1 or 2 ring members which are ═N— atoms (where the remainder of the 6 ring members are carbon atoms).

Bicyclic heteroaromatic groups include fused-ring heteroaromatic groups containing 9-14 ring members wherein 1, 2, 3, 4 or more ring members are independently selected from O, S, N, and NR^{N}.

9-Membered bicyclic heteroaryl groups may contain 1 ring member which is an —NR^{N}— group, an —O— atom or an —S— atom and, optionally, 1-3 ring members (e.g. 1 or 2 ring members) which are ═N— atoms (where the remainder of the 9 ring members are carbon atoms).

Examples of 9-membered fused-ring bicyclic heteroaryl groups are benzofuranyl, benzothiophenyl, indolyl, benzimidazolyl, indazolyl, benzotriazolyl, pyrrolo[2,3-b]pyridinyl, pyrrolo[2,3-c]pyridinyl, pyrrolo[3,2-c]pyridinyl, pyrrolo[3,2-b]pyridinyl, imidazo[4,5-b]pyridinyl, imidazo[4,5-c]pyridinyl, pyrazolo[4,3-d]pyridinyl, pyrazolo[4,3-c]pyridinyl, pyrazolo[3,4-c]pyridinyl, pyrazolo[3,4-b]pyridinyl, isoindolyl, indazolyl, purinyl, indolininyl, imidazo[1,2-a]pyridinyl, imidazo[1,5-a]pyridinyl, pyrazolo[1,2-a]pyridinyl, pyrrolo[1,2-b]pyridazinyl and imidazo[1,2-c]pyrimidinyl.

10-Membered bicyclic heteroaryl groups may contain 1-3 ring members which are ═N— atoms (where the remainder of the 10 ring members are carbon atoms).

Examples of 10-membered fused-ring bicyclic heteroaryl groups are quinolinyl, isoquinolinyl, cinnolinyl, quinazolinyl, quinoxalinyl, phthalazinyl, 1,6-naphthyridinyl, 1,7-naphthyridinyl, 1,8-naphthyridinyl, 1,5-naphthyridinyl, 2,6-naphthyridinyl, 2,7-naphthyridinyl, pyrido[3,2-d]pyrimidinyl, pyrido[4,3-d]pyrimidinyl, pyrido[3,4-d]pyrimidinyl, pyrido[2,3-d]pyrimidinyl, pyrido[2,3-b]pyrazinyl, pyrido[3,4-b]pyrazinyl, pyrimido[5,4-d]pyrimidinyl, pyrazino[2,3-b]pyrazinyl and pyrimido[4,5-d]pyrimidinyl.

The term “heteroarylalkyl” means alkyl substituted with a heteroaryl group.

Examples of acyl groups include alkyl-C(═O)—, cycloalkyl-C(═O)—, alkenyl-C(═O)—, cycloalkenyl-C(═O)—, heteroalkyl-C(═O)—, heterocycloalkyl-C(═O)—, aryl-C(═O)— or heteroaryl-C(═O)—, in particular, alkyl-C(═O)— and aryl-C(═O)—.

Unless indicated explicitly otherwise, where combinations of groups are referred to herein as one moiety, e.g. arylalkyl, the last mentioned group contains the atom by which the moiety is attached to the rest of the molecule.

Where reference is made to a carbon atom of an alkyl group or other group being replaced by O, S(O)ₜ or N, what is intended is that:

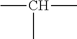

is replaced by

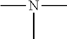

—CH═ is replaced by —N═;
≡C—H is replaced by ≡N; or
—CH₂— is replaced by —O—, —S(O)ₜ— or —NR^{N}—.

By way of clarification, in relation to the above mentioned heteroatom containing groups (such as heteroalkyl etc.), where a numerical of carbon atoms is given, for instance C₃₋₆heteroalkyl, what is intended is a group based on C₃₋₆alkyl in which one of more of the 3-6 chain carbon atoms is replaced by O, S(O)ₜ or N. Accordingly, a C₃₋₆heteroalkyl group, for example, will contain less than 3-6 chain carbon atoms.

Where mentioned above, R^{N} is H, alkyl, cycloalkyl, aryl, heteroaryl, —C(O)-alkyl, —C(O)-aryl, —C(O)-heteroaryl, —S(O)ₜ-alkyl, —S(O)ₜ-aryl or —S(O)ₜ-heteroaryl. R^{N} may, in particular, be H, alkyl (e.g. C₁₋₆alkyl) or cycloalkyl (e.g. C₃₋₆cycloalkyl).

Where mentioned above, t is independently 0, 1 or 2, for example 2. Typically, t is 0.

Where a group has at least 2 positions which may be substituted, the group may be substituted by both ends of an alkylene or heteroalkylene chain to form a cyclic moiety.

Optionally substituted groups (e.g. alkyl, cycloalkyl, alkoxy, alkenyl, cycloalkenyl, alkynyl, alkylene, alkenylene, heteroalkyl, heterocycloalkyl, heteroalkenyl, heterocycloalkenyl, heteroalkynyl, heteroalkylene, heteroalkenylene, aryl, arylalkyl, arylheteroalkyl, heteroaryl, heteroarylalkyl or heteroarylheteroalkyl groups etc.) may be substituted or unsubstituted, or may be unsubstituted. Typically, substitution involves the notional replacement of a hydrogen atom with a substituent group, or two hydrogen atoms in the case of substitution by ═O.

Where substituted, there will generally be 1 to 3 substituents, or 1 or 2 substituents, or 1 substituent.

The optional substituent(s) is/are independently halogen, trihalomethyl, trihaloethyl, —OH, —NH₂, —NO₂, —CN, —N⁺(C₁₋₆alkyl)₂O⁻, —CO₂H, —CO₂C₁₋₆alkyl, —SO₃H, —SOC₁₋₆alkyl, —SO₂C₁₋₆alkyl, —SO₃C₁₋₆alkyl, —OC(═O)OC₁₋₆alkyl, —C(═O)H, —C(═O)C₁₋₆alkyl, —OC(═O)C₁₋₆alky), ═O, —NH(C₁₋₆alkyl), —N(C₁₋₆alkyl)₂, —C(═O)NH₂, —C(═O)N(C₁₋₆alkyl)₂, —N(C₁₋₆alkyl)C(═O)O(C₁₋₆ alkyl), —N(C₁₋₆alkyl)C(═O)N(C₁₋₆alkyl)₂, —OC(═O)N(C₁₋₆alkyl)₂, —N(C₁₋₆alkyl)C(═O)C₁₋₆alkyl, —C(═S)N(C₁₋₆alkyl)₂, —N(C₁₋₆alkyl)C(═S)C₁₋₆alkyl, —SO₂N(C₁₋₆alkyl)₂, —N(C₁₋₆alkyl)SO₂C₁₋₆alkyl, —N(C₁₋₆alkyl)C(═S)N(C₁₋₆alkyl)₂, —N(C₁₋₆alkyl)SO₂N(C₁₋₆alkyl)₂, —C₁₋₆alkyl, —C₁₋₆heteroalkyl, —C₃₋₆cycloalkyl, —C₃₋₆heterocycloalkyl, —C₂₋₆alkenyl, —C₂₋₆heteroalk enyl, —C₃₋₆cycloalkenyl, —C₃₋₆heterocycloalkenyl, —C₂₋₆alkynyl, —C₂₋₆heteroalkynyl, —Z^{u}—C₁₋₆alkyl, —Z^{u}—C₃₋₆cycloalkyl, —Z^{u}—C₂₋₆alkenyl, —Z^{u}—C₃₋₆cycloalkenyl or —Z^{u}—C₂₋₆alkynyl, wherein Z^{u} is independently O, S, NH or N(C₁₋₆alkyl).

In another embodiment, the optional substituent(s) is/are independently halogen, trihalomethyl, trihaloethyl, —NO₂, —CN, —N⁺(C₁₋₆alkyl)₂O—, —CO₂H, —SO₃H, —SOC₁₋₆alkyl, —SO₂C₁₋₆alkyl, —C(═O)H, —C(═O)C₁₋₆alkyl, ═O, —N(C₁₋₆alkyl)₂, —C(═O)NH₂, —C₁₋₆alkyl, —C₃₋₆cycloalkyl, —C₃₋₆heterocycloalkyl, —Z^{u}C₁₋₆alkyl or -z^{u}-C₃₋₆cycloalkyl, wherein Z^{u} is defined above.

In another embodiment, the optional substituent(s) is/are independently halogen, trihalomethyl, —NO₂, —CN, —CO₂H, —C(═O)C₁₋₆alkyl, ═O, —N(C₁₋₆alkyl)₂, —C(═O)NH₂, —C₁₋₆alkyl, —C₃₋₆cycloalkyl, —C₃₋₆heterocycloalkyl, —Z^{u}C₁₋₆alkyl or —Z^{u}—C₃₋₆cycloalkyl, wherein Z^{u} is defined above.

In another embodiment, the optional substituent(s) is/are independently halogen, —NO₂, —CN, —CO₂H, ═O, —N(C₁₋₆alkyl)₂, —C₁₋₆alkyl, —C₃₋₆cycloalkyl or —C₃₋₆heterocycloalkyl.

In another embodiment, the optional substituent(s) is/are independently halogen, —OH, NH₂, NH(C₁₋₆alkyl), —N(C₁₋₆alkyl)₂, —C₁₋₆alkyl, —C₃₋₆cycloalkyl or —C₃₋₆heterocycloalkyl.

The term “polyalkylene glycol” (PAG) refers to compounds having the general formula H—[O—C_{y}H_{2y}]ₓ—OH, such as H—[O—CH₂—CH₂]ₓ—OH (polyethylene glycol or PEG) and H—[O—CH(CH₃)—CH₂]ₓ—OH (polypropylene glycol). When found in a compound of the invention (i.e., in a PAG-ylated polymer of Formula III, of Formula IIIa, or of Formula Ia) the PAG is bound by the bond between a carbon atom and one of the terminal hydroxyl groups e.g. in the case of PEG (i.e., in a PEG-ylated polymer of Formula III, of Formula IIIa, or of Formula Ia) the substituent would be H—[O—CH₂—CH₂]ₓ—. The polyalkylene glycols used in the compounds of the invention, unless otherwise defined, may have a molecular weight of from 500 to 20,000 g/mol, preferably from 1,000 to 10,000 g/mol, more preferably from 2,000 to 5,000 g/mol, more preferably from 2,000 to 3,500 g/mol.

As used herein, the term “polymer of Formula III”, “polymer of Formula IIIa”, or “polymer of Formula Ia” includes pharmaceutically acceptable derivatives thereof and polymorphs, isomers and isotopically labelled variants thereof.

The term “pharmaceutically acceptable derivative” includes any pharmaceutically acceptable salt, solvate, hydrate or prodrug of a polymer of Formula III, of Formula IIIa, or of Formula Ia. The pharmaceutically acceptable derivatives suitably refers to pharmaceutically acceptable salts, solvates or hydrates of a polymer of Formula III, of Formula IIIa, or of Formula Ia. The term “pharmaceutically acceptable salt” includes a salt prepared from pharmaceutically acceptable non-toxic acids or bases including inorganic or organic acids and bases.

Polymers of Formula III, of Formula IIIa, or of Formula Ia which contain basic, e.g. amino, groups are capable of forming pharmaceutically acceptable salts with acids. Pharmaceutically acceptable acid addition salts of the polymers of Formula III, of Formula IIIa, or of Formula Ia may include, but are not limited to, those of inorganic acids such as hydrohalic acids (e.g. hydrochloric, hydrobromic and hydroiodic acid), sulfuric acid, nitric acid and phosphoric acids. Pharmaceutically acceptable acid addition salts of the polymers of Formula III, of Formula IIIa, or of Formula Ia may include, but are not limited to, those of organic acids such as aliphatic, aromatic, carboxylic and sulfonic classes of organic acids, examples of which include: aliphatic monocarboxylic acids such as formic acid, acetic acid, propionic acid or butyric acid; aliphatic hydroxy acids such as lactic acid, citric acid, tartaric acid or malic acid; dicarboxylic acids such as maleic acid or succinic acid; aromatic carboxylic acids such as benzoic acid, p-chlorobenzoic acid, phenylacetic acid, diphenylacetic acid or triphenylacetic acid; aromatic hydroxyl acids such as o-hydroxybenzoic acid, p-hydroxybenzoic acid, 1-hydroxynaphthalene-2-carboxylic acid or 3-hydroxynaphthalene-2-carboxylic acid; and sulfonic acids such as methanesulfonic acid, ethanesulfonic acid or benzenesulfonic acid. Other pharmaceutically acceptable acid addition salts of the polymers of Formula III, of Formula IIIa, or of Formula Ia include, but are not limited to, those of glycolic acid, glucuronic acid, furoic acid, glutamic acid, anthranilic acid, salicylic acid, mandelic acid, embonic (pamoic) acid, pantothenic acid, stearic acid, sulfanilic acid, algenic acid and galacturonic acid. Wherein the polymer of Formula III, of Formula IIIa, or of Formula Ia comprises a plurality of basic groups, multiple centres may be protonated to provide multiple salts, e.g. di- or tri-salts of compounds of Formula III, of Formula IIIa, or of Formula Ia. For example, a hydrohalic acid salt of a polymer of Formula III, of Formula IIIa, or of Formula Ia as described herein may be a monohydrohalide, dihydrohalide or trihydrohalide, etc. The salts include, but are not limited to those resulting from addition of any of the acids disclosed above. In one embodiment of the polymer of Formula III, of Formula IIIa, or of Formula Ia, two basic groups form acid addition salts. In a further embodiment, the two addition salt counterions are the same species, e.g. dihydrochloride, dihydrosulphide etc. Typically, the pharmaceutically acceptable salt is a hydrochloride salt, such as a dihydrochloride salt.

Polymers of Formula III, of Formula IIIa, or of Formula Ia which contain acidic, e.g. carboxyl, groups are capable of forming pharmaceutically acceptable salts with bases. Pharmaceutically acceptable basic salts of the polymers of Formula III, of Formula IIIa, or of Formula Ia may include, but are not limited to, metal salts such as alkali metal or alkaline earth metal salts (e.g. sodium, potassium, magnesium or calcium salts) and zinc or aluminium salts. Pharmaceutically acceptable basic salts of the polymers of Formula III, of Formula IIIa, or of Formula Ia may include, but are not limited to, salts formed with ammonia or pharmaceutically acceptable organic amines or heterocyclic bases such as ethanolamines (e.g. diethanolamine), benzylamines, N-methyl-glucamine, amino acids (e.g. lysine) or pyridine. Hemisalts of acids and bases may also be formed, e.g. hemisulphate salts.

Pharmaceutically acceptable salts of polymers of Formula III, of Formula IIIa, or of Formula Ia may be prepared by methods well-known in the art. For a review of pharmaceutically acceptable salts, see Stahl and Wermuth, Handbook of Pharmaceutical Salts: Properties, Selection and Use (Wiley-VCH, Weinheim, Germany, 2002).

The polymers of Formula III, of Formula IIIa, or of Formula Ia may exist in both unsolvated and solvated forms. The term “solvate” includes molecular complexes comprising the polymer and one or more pharmaceutically acceptable solvent molecules such as water or C₁₋₆ alcohols, e.g. ethanol. The term “hydrate” means a “solvate” where the solvent is water.

The polymers may exist in solid states from amorphous through to crystalline forms. All such solid forms are included within the invention.

The polymers may exist in one or more geometrical, optical, enantiomeric, diastereomeric and tautomeric forms, including but not limited to cis- and trans-forms, E- and Z-forms, R-, S- and meso-forms, keto- and enol-forms. All such isomeric forms are included within the invention. The isomeric forms may be in isomerically pure or enriched form, as well as in mixtures of isomers (e.g. racemic or diastereomeric mixtures).

The invention includes pharmaceutically acceptable isotopically-labelled polymers of Formula III, of Formula IIIa, or of Formula Ia wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled polymers of Formula III, of Formula IIIa, or of Formula Ia, for example, those incorporating a radioactive isotope, are useful in drug and/or substrate tissue distribution studies. The radioactive isotopes ³H and ¹⁴C are particularly useful for this purpose in view of their ease of incorporation and ready means of detection. Substitution with positron emitting isotopes, such as ¹¹C, ¹⁸F, ¹⁵O and ¹³N, can be useful in Positron Emission Topography (PET) studies for examining substrate receptor occupancy.

Isotopically-labelled polymers of Formula III, of Formula IIIa, or of Formula Ia can generally be prepared by conventional techniques known to those skilled in the art or by processes analogous to those described herein using an appropriate isotopically-labelled reagent in place of the non-labelled reagent previously employed.

It will be appreciated that the polymers, as described herein, may be substituted with any number of substituents or functional moieties. The terms substituted, whether preceded by the term “optionally” or not, and substituent, as used herein, refer to the ability, as appreciated by one skilled in this art, to change one functional group for another functional group provided that the valency of all atoms is maintained. When more than one position in any given structure may be substituted with more than one substituent selected from a specified group, the substituent may be either the same or different at every position. The substituents may also be further substituted (e.g., an aryl group substituent may have another substituent off it, such as another aryl group, which is further substituted with fluorine at one or more positions). The term thiohydroxyl or thiol, as used herein, refers to a group of the formula —SH.

### Virus-Based Therapeutic Agents

The virus-based therapeutic agent is an AAV vector suitable for use in therapy. In some embodiments, the AAV vector is suitable for use in systemic viral gene therapy. In some embodiments, the AAV vector is an oncolytic viral vector. In some embodiments, the AAV vector is for use in a vaccine.

### Covalently Coated AAV Vector

Throughout the description and claims the word “comprise” and variations of the word, are not intended to exclude other technical features, additives, components, or steps.

Furthermore, the word “comprise” encompasses the case of “consisting of”. Furthermore, the present invention covers all possible combinations of particular and preferred embodiments described herein.

The following examples and drawings are provided by way of illustration, and they are not intended to be limiting of the present invention.

### EXAMPLES

Mouse motor neuron-like hybrid cell line (NSC-34), mouse myoblast cell line (02012), primary vascular aortic smooth muscle cells (MOVAS), and adeno-associated virus 9 (AAV9)-green fluorescent protein (GFP) are commercially available.

PBAE polymers were prepared as disclosed in WO2018215488 (see Example 1 for the synthesis of PBAE polymers; and Example 2A for the synthesis of acrylate terminated intermediate (C6)). Two different polymers were synthesized, one containing three types of monomers and an oligopeptide at both ends and another one combining the three monomers with a fourth one containing a PEG moiety.

### Example 1—Synthesis of an Acid Activated OM-PBAE

To achieve a covalent coating, an acid activated OM-PBAE such as R3C—C6-Act-CR3 polymer was obtained by first bonding an activated acid such as the succinic acid derivative of the NHS to a PBAE molecule such as C6 PBAE

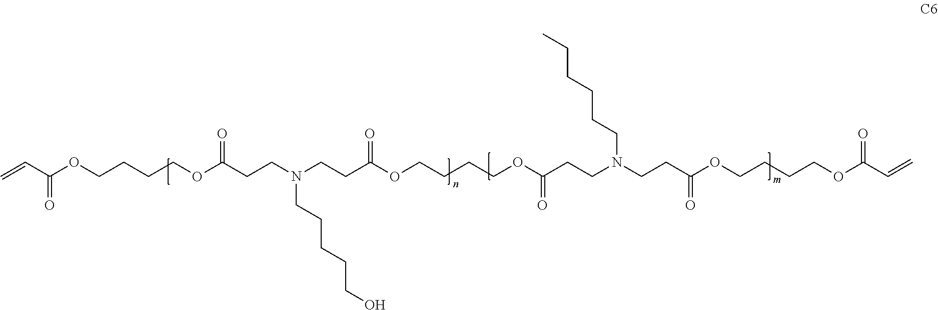

and afterwards, attaching to the activated PBAE a peptide such as Cys-Arg-Arg-Arg.

### Step 1. Synthesis of the Succinic Acid Derivative of the N-hydroxysuccinimide

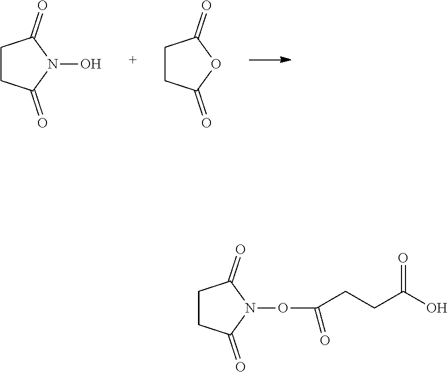 [SMILES: O=C1CCC(=O)N1O ; O=C1CCC(=O)O1 ; O=C(O)CCC(=O)ON1C(=O)CCC1=O]

To obtain the succinic acid derivative of the NHS, a 10 mL round-bottom flask, 0.5 mmol of succinic anhydride, 2.5 mL of dichloromethane and 0.5 mmol of N-hydroxysuccinimide were mixed. The mixture was sonicated for 5 minutes and it is then stirred in a water bath with a controlled temperature of 25° C.±2° C. for 4 hours. The solvent was then evaporated in a rotary evaporator at 40° C. and a pressure of 800 mbar. The product was kept at 4° C. until further use.

Step 2. Synthesis of C6 PBAE Activated with the Succinic Acid Derivative of N-hydroxysuccinimide

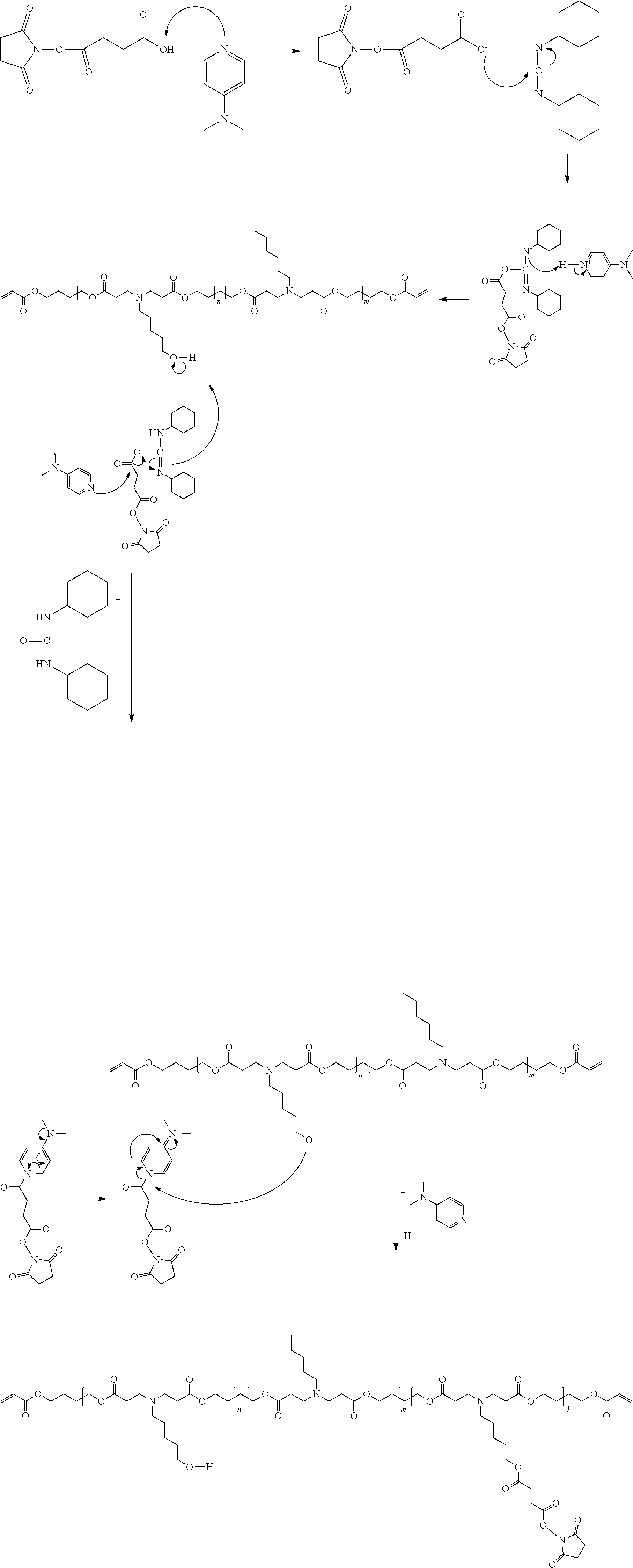

The acrylate terminated intermediate (C6) was prepared as disclosed in Example 2A of EP3406265B1.

In a screw thread glass vial, 0.095 mmol of C6 PBAE, 2.5 mL of dichloromethane and 0.095 mmol of the succinic acid derivative of the NHS were introduced and stirred. Agitation was maintained throughout the reaction. The solution was cooled at 0° C. and subsequently, 0.1 mmol of N,N′-dicyclohexylcarbodiimide and 0.01 mmol of 4-dimethylaminopyridine were added. After 15 minutes, the mixture was introduced in a water bath with a controlled temperature of 25° C.±2° C. for 18 hours. The mixture was cooled for 3 hours. The solid was then filtered off with a syringe and a 0.45 μm filter, and 0.02 mL of a 1 mg/mL solution of hydroquinone in diethyl ether were added (to avoid the polymerization of the acrylates in the following step). The solvent was evaporated in a rotary evaporator at 40° C. and a pressure of 800 mbar.

The polymer was diluted in 1 mL of 1:2 (v/v) acetonitrile/ethyl acetate, the solution was cooled for 20 minutes and it was filtered using a syringe and a 0.45 μm filter. The solvent was evaporated in a rotary evaporator at 40° C. and a pressure of 200 mbar.

### Step 3. Synthesis of R3C—C6-Act-CR3 Polymer

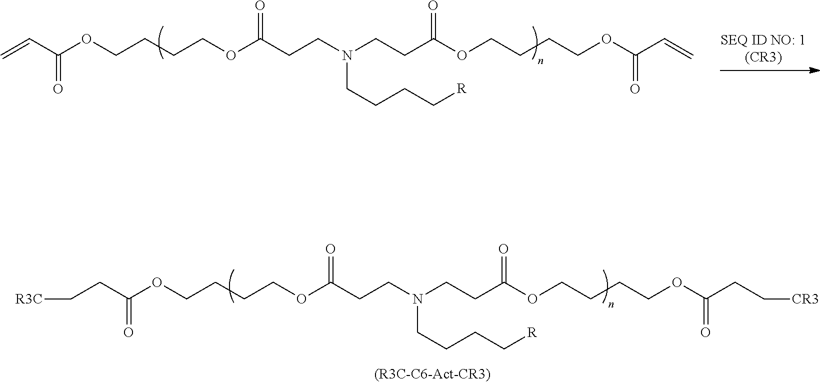

wherein
R: independently selected from

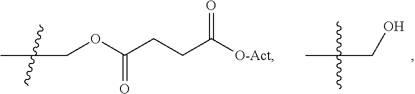

and CH₃;
“Act” is N-succinimidyl, and at least one R is

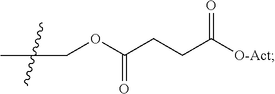

at least one R is

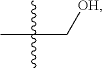 [SMILES: *CO]

and at least one R is CH₃;
and R3C— or —CR3 (SEQ ID NO: 2) is:

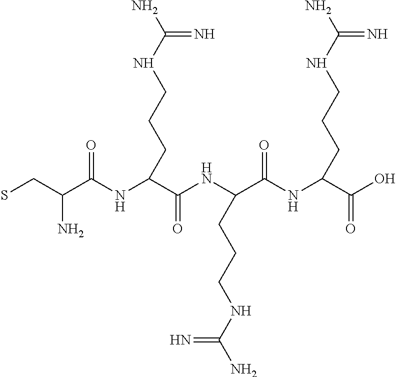 [SMILES: N=C(N)NCCCC(NC(=O)C(CCCNC(=N)N)NC(=O)C(CCCNC(=N)N)NC(=O)C(N)CS)C(=O)O]

that is, the cysteine is linked to the PBAE (C6) by the thiol group.

Once the one-acid activated PBAE (particularly, the succinic acid derivative of C6 PBAE activated with N-hydroxysuccinimide) was synthesized, the CR3 peptide (SEQ ID NO: 1=Cys-Arg-Arg-Arg) was attached. A solution of 0.054 mmol of PBAE in 1.1 mL of dimethyl sulfoxide and another solution of 0.13 mmol of the hydrochloride of CR3 in 1 mL of dimethyl sulfoxide were prepared. Both solutions were mixed in a screw cap tube and the mixture solution was stirred in a water bath with a controlled temperature of 25° C.±2° C. for 20 hours. The mixture was added dropwise to 7:3 (v/v) diethyl ether/acetone (8 mL). The resulting suspension was centrifuged at 4000 rpm for 10 minutes and the solvent was removed. The solid was washed twice with 7:3 (v/v) diethyl ether/acetone (2×4 mL). The product was dried for 24 hours in a vacuum oil pump. A 100 mg/mL solution of the product in dimethyl sulfoxide was made and it was kept refrigerated at −20° C. until further use.

### Example 2—Synthesis of an Acid Activated OM-PBAE-PEG

A C6-PEG PBAE activated with N-hydroxysuccinimide was obtained following the same procedure of Example 1, but for the use of C6-PEG PBAE instead of C6 PBAE in order to obtain a R3C—C6-Act-PEG-CR3 polymer.

### Example 3—Synthesis and Characterization of the Covalently Coated AAV Vector

The physicochemical properties of the AAV covalently coated with the R3C—C6-Act-CR3 polymer or the combined R3C—C6-Act-CR3 and R3C—C6-Act-PEG-CR3 polymers were characterized as compared to the electrostatically coated AAV.

### Synthesis of the AAV Covalently Coated Particles—Surface Charge Change

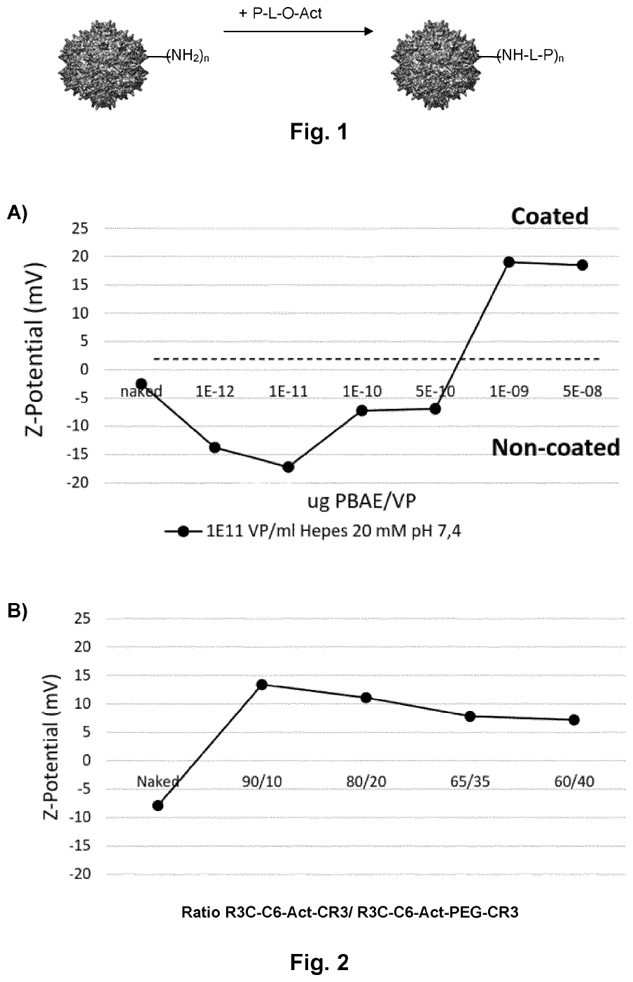

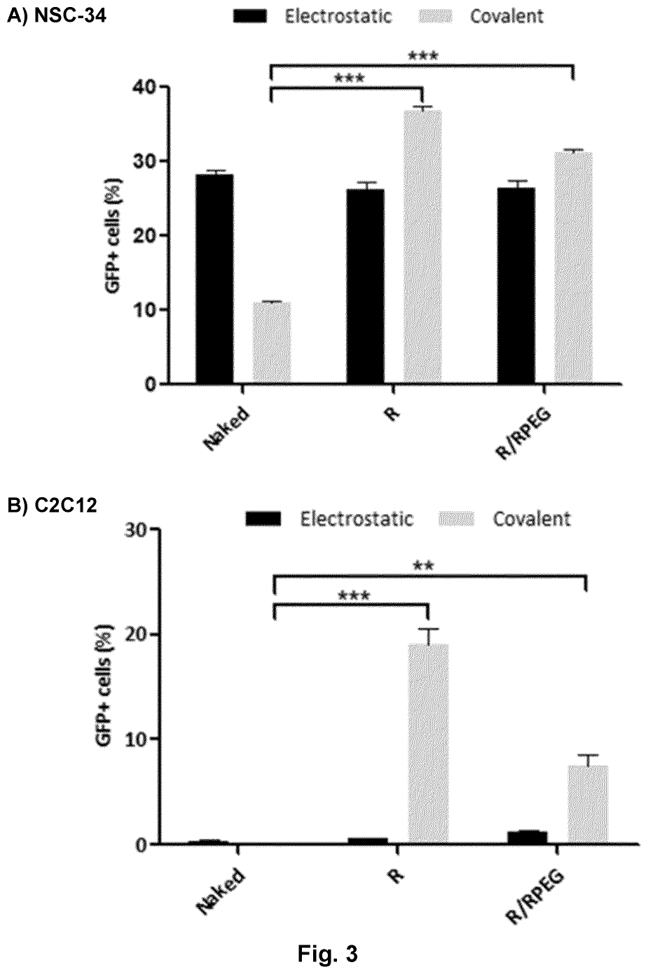

1E11 vp of AAV were mixed with increasing concentrations of the polymer, and the surface charges were analyzed by dynamic light scattering (DLS) measuring the Z-potential. Changes in the surface charge of coated particles reached a plateau at 1E-9 μg of polymer/vp (FIG. 2, A), 1E-8 ratio was chosen to continue with the characterization. It is important to note that it is necessary to realize the coating in Hepes 20 mM at a pH of 7.4 (FIG. 1A).

In order to increase the stability, to decrease the aggregation and to increase the circulating time of the nanoformulations, a similar synthesis was carried out by additionally adding polyethylene glycol (PEG) to the formulation. Thus, nanoparticles at a 1E-8 μg of polymer/vp ratio combining the polymers R3C—C6-Act-CR3 and R3C—C6-Act-PEG-CR3 at different percentages were prepared. The Z-potential at different ratios of R3C—C6-Act-CR3/R3C—C6-Act-PEG-CR3 (from 90/10 to 60/40) was measured and positive values in all the conditions were obtained. As expected, a decrease in the surface charge was observed while the R3C—C6-Act-PEG-CR3 percentage was increased (FIG. 2B).

The results demonstrate that R3C—C6-Act-CR3/R3C—C6-Act-PEG-CR3 polymers successfully covalently bonded to viral particles, and that the formulation resulted in disperse nanoparticles with positive charge.

### Transduction Efficiency

Mouse motor neuron-like hybrid cell line (R3C), used as a permissive cell line, mouse myoblast cell line (02012) and primary vascular aortic smooth muscle cells (MOVAS) were transduced with both the electrostatic and the covalent coated viral particles in order to study whether they presented any differences in vitro. The electrostatic coating was generated with R3C—C6-CR3 or R3C—C6-CR3/R3C—C6PEG-CR3 (volume ratio of 60/40) polymer in saline buffer, whereas the covalent coating was formed with R or R/RPEG (volum ratio of 60/40) polymer using Hepes buffer. Naked viral particles only contained the corresponding buffer. In all samples, the viral vector used was rAAV9-GFP, and its expression was determined by flow cytometry analysis at 72 h post-transduction. Results of flow cytometry are presented FIG. 2. These results demonstrate that the coated viral particles were able to transduce different muscular cell lines surprisingly better that the electrostatically coated viral particles.

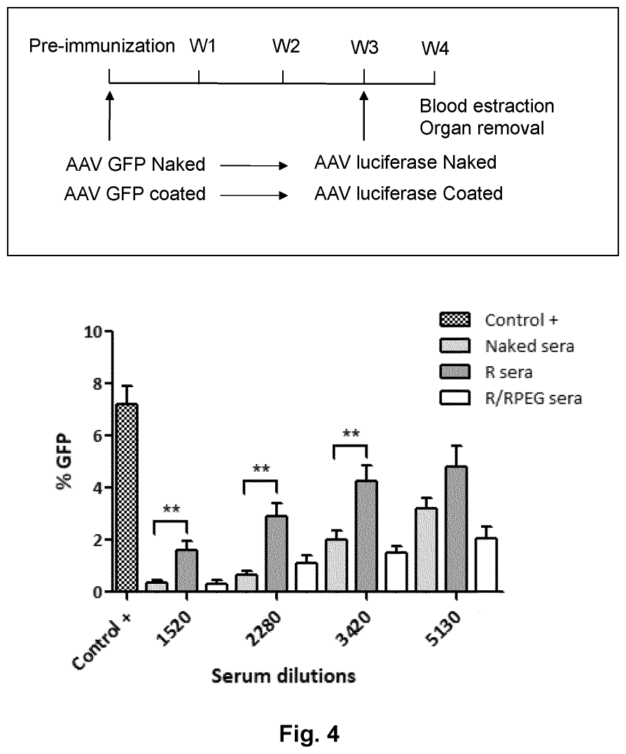

To demonstrate the protection of the coated AAV vectors against the recognition by neutralizing antibodies, 8 Balb/c mice were injected with rAAV9-GFP naked (n=4) or AAV9-GFP coated with R3C—C6-Act-CR3/R3C—C6-Act-PEG-CR3 at a volum ratio of 60/40 (n=4). 3 weeks after the inoculation, serum was purified and 1,5E9 vp/well (MOI: 100.000) were incubated with different dilutions of the extracted serums (FIG. 4). The significant increase in GFP expression in the transfection with the coated viral particles compared with the naked ones, demonstrate the efficient protection from neutralizing antibodies against AAV.

### CITATION LIST

1. EP3406265B1.
2. Brugada, P., “Boosting intravenous administration of therapeutic viral vectors using an oligopeptide-modified poly(p-aminoester)s-based coating technology”, Universitat Ramon Llull, Spain, 2018.
3. Green J J, Langer R, Anderson DG. “A combinatorial polymer library approach yields insight into nonviral gene delivery”. Acc Chem Res. 2008; 41(6):749-759.
4. Green J J, Zhou B Y, Mitalipova M M, et al. “Nanoparticles for gene transfer to human embryonic stem cell colonies”. Nano Lett. 2008; 8(10):3126-3130.

What is claimed is:

1. An adeno-associated virus (AAV) vector particle, wherein the AAV has at least one capsid protein having one N-terminal covalently bond to a poly(beta aminoester) (PBAE), or a pharmaceutically acceptable salt thereof.

2. The AAV vector particle of claim 1, wherein the PBAE is an oligopeptide modified PBAE (OM-PBAE).

3. The AAV vector particle of claim 1, having a structure of Formula (VII)
(A)-(L)-(P) (VII)
wherein:
L is a linker moiety having a first and a second carbonyl groups which is a biradical of formula —C(═O)-L′-C(═O)—, wherein L′ is a biradical selected from the group consisting of alkylene, cycloalkylene, alkenylene, cycloalkenylene, heteroalkylene, heterocycloalkylene, arylene, and heteroarylene group.
P is a radical of the PBAE, or of a pharmaceutically acceptable salt thereof, wherein
the PBAE is
a) a polymer of Formula (III), 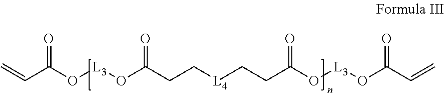
wherein
L₃ is independently selected from the group consisting of alkylene, alkenylene, heteroalkylene, heteroalkenylene, arylene, and heteroarylene; or at least one occurrence of L₃ is 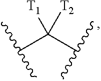
wherein:
T₁ is 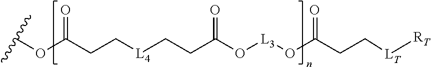
T₂ is selected from the group consisting of H, alkyl, and 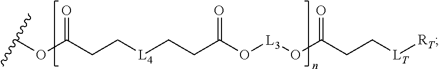
L_{T} is independently selected from the group consisting of: 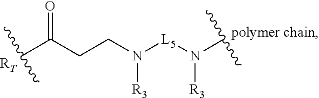
O, S, NRₓ, and a bond, wherein Rₓ is independently selected from the group consisting of hydrogen, halogen, alkyl, cycloalkyl, alkenyl, cycloalkenyl, heteroalkyl, heterocycloalkyl, acyl, aryl, and heteroaryl, and
the remaining L₃ groups are independently selected at each occurrence from the group consisting of alkylene, alkenylene, heteroalkylene, heteroalkenylene, arylene, and heteroarylene;
L₄ is independently selected from the group consisting of 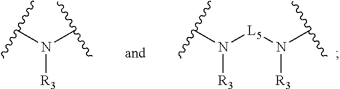
L₅ is independently selected from the group consisting of alkylene, alkenylene, heteroalkylene, heteroalkenylene, arylene, and heteroarylene;
R_{T} is independently selected from an oligopeptide and R_{y};
and wherein R_{y} is selected from the group consisting of hydrogen, halogen, alkyl, cycloalkyl, alkenyl, cycloalkenyl, heteroalkyl, heterocycloalkyl, acyl, aryl, and heteroaryl;
each R₃ is independently selected from the group consisting of hydrogen, halogen, alkyl, cycloalkyl, alkenyl, cycloalkenyl, heteroalkyl, heterocycloalkyl, acyl, aryl, heteroaryl, and polyalkylene glycols, wherein said polyalkylene glycol is either bound directly to the nitrogen atom to which R₃ is attached or bound to the nitrogen atom to which R₃ is attached via a linker moiety, wherein said linker moiety is an alkylene, cycloalkylene, alkenylene, cycloalkenylene, heteroalkylene, heterocycloalkylene, arylene or heteroarylene group;
provided that at least one R₃ is a moiety having a hydroxyl group (—OH) or an amino group (—NH₂) independently selected from the group consisting of —(CH₂)ₚNH₂, —(CH₂)ₚOH, —(CH₂)₂(OCH₂CH₂)_{q}NH₂, and —(CH₂)₂(OCH₂CH₂)_{q}OH;
n is an integer from 5 to 1,000, p is an integer from 1 to 20, and q is an integer from 1 to 10;
or alternatively
b) a polymer of Formula (I) 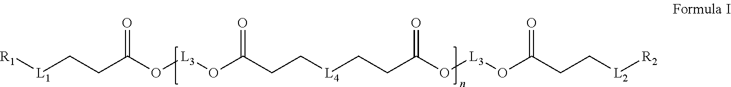
wherein L₃, L₄, and n are as defined above for polymer of Formula III, and
each L₁ and L₂ is independently selected from the group consisting of: 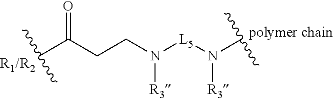
O, S, NRₓ, and a bond; wherein Rₓ is independently selected from the group consisting of hydrogen, halogen, alkyl, cycloalkyl, alkenyl, cycloalkenyl, heteroalkyl, heterocycloalkyl, acyl, aryl, and heteroaryl; each R₃″ is independently selected from the group consisting of hydrogen, halogen, alkyl, cycloalkyl, alkenyl, cycloalkenyl, heteroalkyl, heterocycloalkyl, acyl, aryl, heteroaryl, and polyalkylene glycols, wherein said polyalkylene glycol is either bound directly to the nitrogen atom to which R₃″ is attached or bound to the nitrogen atom to which R₃″ is attached via a linker moiety, wherein said linker moiety is an alkylene, cycloalkylene, alkenylene, cycloalkenylene, heteroalkylene, heterocycloalkylene, arylene or heteroarylene group; and L₅ is as defined above;
R₁ and R₂ and R_{T} are independently selected from an oligopeptide and R_{y}; wherein at least one of R₁ and R₂ and R_{T} is an oligopeptide; wherein R_{y} is as defined above; and
wherein P is attached to the linker L by the hydroxyl or the amino group of the PBAE of Formula (III) or of Formula (I);
A is a radical of an AAV having at least one capsid protein having one N-terminal, wherein A is attached to the linker L by the N-terminal; and wherein
L is attached to the radical P through a bond which is an ester or an amide bond, the ester bond being formed between the hydroxyl group of the polymer of Formula III, or alternatively the hydroxyl group of the polymer of Formula I and one carbonyl group of the linker L; or the amide bond being formed between the amino group of the polymer of Formula III or the amino group of the polymer of Formula I and the one group of the linker L; and
L is attached to A through an amide bond formed between the N-terminal of the at least one capsid protein and the other one carbonyl group of the linker L.

4. The AAV vector particle of claim 3, wherein L′ is a —(CH₂)ᵣ— biradical, wherein r is an integer independently selected from 1 to 5.

5. The AAV vector particle of claim 3, wherein the at least one R₃ is independently selected from —(CH₂)ₚNH₂ and —(CH₂)ₚOH, wherein p is an integer from 1 to 5, and q is an integer from 1 to 5.

6. The AAV vector particle of claim 3, wherein at least one R₃ or at least one R₃″ group is a polyalkylene glycol.

7. The AAV vector particle of claim 3, wherein (i) the at least one R₃ or at least one R₃″ group which is a polyalkylene glycol is bound to the nitrogen atom to which it is attached through a linker moiety which is an alkylene, alkenylene or heteroalkylene group; or (ii) the at least one R₃ or at least one R₃″ group which is a polyalkylene glycol is bound directly to the nitrogen atom to which it is attached.

8. The AAV vector particle of claim 3, wherein the or each oligopeptide is a radical of Formula VII: 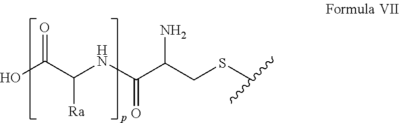
wherein p is an integer from 2 to 19 and wherein Rₐ is selected at each occurrence from the group consisting of H₂NC(═NH)—NH(CH₂)₃—, H₂N(CH₂)₄—, and (1H-imidazol-4-yl)-CH₂—.

9. A pharmaceutical composition comprising the AAV vector particle as defined in claim 1, together with a pharmaceutically acceptable vehicle.

10. An acid activated PBAE which is a polymer of formula IIIa: 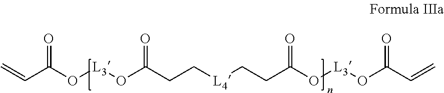
wherein L₃′ is independently selected from the group consisting of alkylene, alkenylene, heteroalkylene, heteroalkenylene, arylene, and heteroarylene; or at least one occurrence of L₃′ is 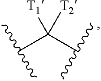
wherein T₁′ is 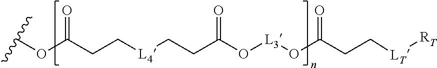
and T₂′ is selected from H, alkyl or 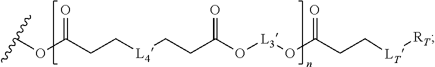
wherein L_{T}′ is independently selected from the group consisting of: 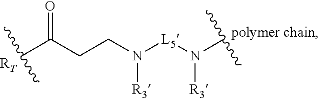
O, S, NRₓ and a bond, wherein Rₓ is independently selected from the group consisting of hydrogen, halogen, alkyl, cycloalkyl, alkenyl, cycloalkenyl, heteroalkyl, heterocycloalkyl, acyl, aryl, and heteroaryl,
and the remaining L₃′ groups are independently selected at each occurrence from the group consisting of alkylene, alkenylene, heteroalkylene, heteroalkenylene, arylene, and heteroarylene;
L₄′ is independently selected from the group consisting of 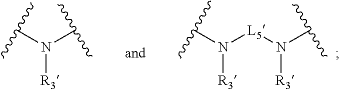
L₅′ is independently selected from the group consisting of alkylene, alkenylene, heteroalkylene, heteroalkenylene, arylene, and heteroarylene;
R_{T} is independently selected from an oligopeptide and R_{y};
and wherein R_{y} is selected from the group consisting of hydrogen, halogen, alkyl, cycloalkyl, alkenyl, cycloalkenyl, heteroalkyl, heterocycloalkyl, acyl, aryl, and heteroaryl;
each R₃′ is independently selected from the group consisting of hydrogen, halogen, alkyl, cycloalkyl, alkenyl, cycloalkenyl, heteroalkyl, heterocycloalkyl, acyl, aryl, heteroaryl, and polyalkylene glycols, wherein said polyalkylene glycol is either bound directly to the nitrogen atom to which R₃′ is attached or bound to the nitrogen atom to which R₃′ is attached via a linker moiety, wherein said linker moiety is an alkylene, cycloalkylene, alkenylene, cycloalkenylene, heteroalkylene, heterocycloalkylene, arylene or heteroarylene group; provided that at least one R₃′ is a moiety selected from the group consisting of —(CH₂)ₚNH—C(═O)-L′-C(═O)—O-Act, —(CH₂)ₚO—C(═O)-L′-C(═O)—O-Act,
—(CH₂)₂(OCH₂CH₂)_{q}NH—C(═O)-L′-C(═O)—O-Act, and
—(CH₂)₂(OCH₂CH₂)_{q}O—C(═O)-L′-C(═O)—O-Act, wherein the moiety —C(═O)—O-Act is an activated carboxyl group and Act is an electron withdrawing moiety, or, alternatively, at least one R₃′ is a moiety selected from the group consisting of
—(CH₂)ₚNH—C(═O)-L′-C(═O)-Act′, —(CH₂)ₚO—C(═O)-L′-C(═O)-Act′,
—(CH₂)₂(OCH₂CH₂)_{q}NH—C(═O)-L′-C(═O)-Act′, and
—(CH₂)₂(OCH₂CH₂)_{q}O—C(═O)-L′-C(═O)-Act′, wherein the moiety —C(═O)-Act′ is an activated carboxyl group and Act′ is an electron withdrawing moiety; wherein L′ is a biradical selected from the group consisting of alkylene, cycloalkylene, alkenylene, cycloalkenylene, heteroalkylene, heterocycloalkylene, arylene, and heteroarylene group; and
n is an integer from 5 to 1,000, p is an integer from 1 to 20, and q is an integer from 1 to 10;
or a pharmaceutically acceptable salt thereof.

11. An acid activated OM-PBAE which is a polymer of Formula Ia: 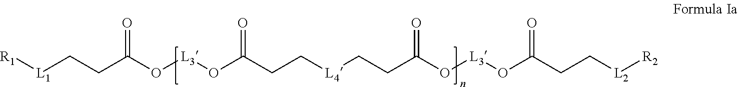
wherein L₃′ is independently selected from the group consisting of alkylene, alkenylene, heteroalkylene, heteroalkenylene, arylene, and heteroarylene; or at least one occurrence of L₃′ is 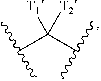
wherein T₁′ is 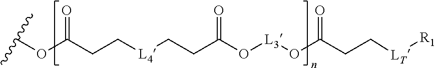
and T₂′ is selected from H, alkyl or 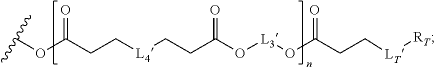
wherein L_{T}′ is independently selected from the group consisting of: 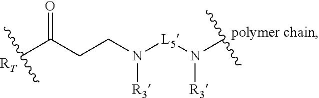
O, S, NRₓ and a bond, wherein Rₓ is independently selected from the group consisting of hydrogen, halogen, alkyl, cycloalkyl, alkenyl, cycloalkenyl, heteroalkyl, heterocycloalkyl, acyl, aryl, and heteroaryl,
and the remaining L₃′ groups are independently selected at each occurrence from the group consisting of alkylene, alkenylene, heteroalkylene, heteroalkenylene, arylene, and heteroarylene; and
L₄′ is independently selected from the group consisting of 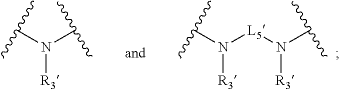
L₅′ is independently selected from the group consisting of alkylene, alkenylene, heteroalkylene, heteroalkenylene, arylene, and heteroarylene;
each R₃′ is independently selected from the group consisting of hydrogen, halogen, alkyl, cycloalkyl, alkenyl, cycloalkenyl, heteroalkyl, heterocycloalkyl, acyl, aryl, heteroaryl, and polyalkylene glycols, wherein said polyalkylene glycol is either bound directly to the nitrogen atom to which R₃′ is attached or bound to the nitrogen atom to which R₃′ is attached via a linker moiety, wherein said linker moiety is an alkylene, cycloalkylene, alkenylene, cycloalkenylene, heteroalkylene, heterocycloalkylene, arylene or heteroarylene group; provided that at least one R₃′ is a moiety selected from the group consisting of —(CH₂)ₚNH—C(═O)-L′-C(═O)—O-Act, —
—(CH₂)ₚO—C(═O)-L′-C(═O)—O-Act,
—(CH₂)₂(OCH₂CH₂)_{q}NH—C(═O)-L′-C(═O)—O-Act, and —(CH₂)₂(OCH₂CH₂)_{q}O—C(═O)-L′-C(═O)—O-Act, wherein the moiety —C(═O)—O-Act is an activated carboxyl group and Act is an electron withdrawing moiety, or, alternatively, at least one R₃′ is a moiety selected from the group consisting of
—(CH₂)ₚNH—C(═O)-L′-C(═O)-Act′, —(CH₂)ₚO—C(═O)-L′-C(═O)-Act′,
—(CH₂)₂(OCH₂CH₂)_{q}NH—C(═O)-L′-C(═O)-Act′, and
—(CH₂)₂(OCH₂CH₂)_{q}O—C(═O)-L′-C(═O)-Act′, wherein the moiety —C(═O)-Act′ is an activated carboxyl group and Act′ is an electron withdrawing moiety; wherein L′ is a biradical selected from the group consisting of alkylene, cycloalkylene, alkenylene, cycloalkenylene, heteroalkylene, heterocycloalkylene, arylene, and heteroarylene group;
each L₁ and L₂ are independently selected from the group consisting of: 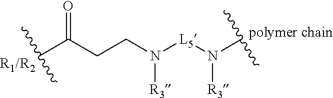
O, S, NRₓ and a bond; wherein Rₓ is independently selected from the group consisting of hydrogen, halogen, alkyl, cycloalkyl, alkenyl, cycloalkenyl, heteroalkyl, heterocycloalkyl, acyl, aryl, and heteroaryl; each R₃″ is independently selected from the group consisting of hydrogen, halogen, alkyl, cycloalkyl, alkenyl, cycloalkenyl, heteroalkyl, heterocycloalkyl, acyl, aryl, heteroaryl, and polyalkylene glycols, wherein said polyalkylene glycol is either bound directly to the nitrogen atom to which R₃″ is attached or bound to the nitrogen atom to which R₃″ is attached via a linker moiety, wherein said linker moiety is an alkylene, cycloalkylene, alkenylene, cycloalkenylene, heteroalkylene, heterocycloalkylene, arylene or heteroarylene group;
R₁ and R₂ and R_{T} are independently selected from an oligopeptide and R_{y}; wherein at least one of R₁ and R₂ and R_{T} is an oligopeptide; wherein R_{y} is selected from the group consisting of hydrogen, halogen, alkyl, cycloalkyl, alkenyl, cycloalkenyl, heteroalkyl, heterocycloalkyl, acyl, aryl, and heteroaryl; and
n is an integer from 5 to 1,000, p is an integer from 1 to 20, and q is an integer from 1 to 10;
or a pharmaceutically acceptable salt thereof.

12. (canceled)

13. A method for the treatment of a systemic viral genetic disease, the method comprising administering the AAV vector particle as defined in claim 1, together with a pharmaceutically acceptable vehicle, to a subject in need thereof.

14. A method for the treatment of cancer, the method comprising administering the AAV vector particle as defined in claim 1, together with a pharmaceutically acceptable vehicle, to a subject in need thereof.

15. A method for the treatment of coronavirus disease 2019 (COVID-19) caused by severe acute respiratory syndrome coronavirus 2 (SARS-CoV-2) or variants thereof, the method comprising administering a viral vector vaccine comprising the AAV vector particle as defined in claim 1, together with a pharmaceutically acceptable vehicle, to a subject in need thereof.

16. The method of claim 14, wherein the cancer is liver cancer or pancreatic cancer.

17. A method for the treatment of a systemic viral genetic disease, the method comprising administering the pharmaceutical composition as defined in claim 9, together with a pharmaceutically acceptable vehicle, in a subject in need thereof.

18. A method for the treatment of cancer, the method comprising administering the pharmaceutical composition as defined in claim 9, together with a pharmaceutically acceptable vehicle, in a subject in need thereof.

19. The method of claim 18, wherein the cancer is liver cancer or pancreatic cancer.

20. A method for the treatment of coronavirus disease 2019 (COVID-19) caused by severe acute respiratory syndrome coronavirus 2 (SARS-CoV-2) or variants thereof, the method comprising administering a viral vector vaccine comprising the pharmaceutical composition as defined in claim 9, together with a pharmaceutically acceptable vehicle, in a subject in need thereof.
